SEQUENCE LISTING (28 sequences)

SEQ ID NO: 1 - protein, 449 aa, Artificial Sequence
  Cetuximab antibody heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Cetuximab antibody light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 15 aa, Artificial Sequence
  Spacer 1
GGGGSGGGGSGGGGS

SEQ ID NO: 4 - protein, 17 aa, Artificial Sequence
  Spacer 2
GPGGGGSGGGGSGGGGS

SEQ ID NO: 5 - protein, 74 aa, Homo sapiens
SAGFFMEELNTYRQKQGVVLKYQELPNSGPPHDRRFTFQVIIDGREFPEGEGRSKKEAKNAAAKLAVEILNKEK

SEQ ID NO: 6 - protein, 73 aa, Homo sapiens
LSMGNYIGLINRIAQKKRLTVNYEQVASGVHGPEGFHYKVKMGQKEYSIGTGSTKQEAKQLAAKLAYLQILSE

SEQ ID NO: 7 - protein, 21 aa, Homo sapiens
KKAVSPLLLTTTNSSEGLSMG

SEQ ID NO: 8 - protein, 168 aa, Homo sapiens
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQVASGVHGPEGFHYKVKMGQKEYSIGTGSTKQEAKQLAAKLAYLQILSE

SEQ ID NO: 9 - DNA, 507 bp, Homo sapiens
atgatggctggtgatctttccgctggcttcttcatggaagagttgaacacctacaggcaaaagcagggcgttgtgctcaagtaccaagagcttccaaattccggaccaccacacgataggcgtttcactttccaggtgatcatcgatggacgtgagttcccagaaggtgagggcagatctaagaaagaggctaagaacgctgctgctaagctcgctgttgagatcctgaacaaagagaagaaggccgtcagtccactcctcctgactactactaattcctccgagggactctccatgggaaactacattggactcatcaacaggatcgcccagaagaagaggctcaccgttaactacgagcaagtggcttctggtgttcatggaccagagggattccactacaaggtgaagatgggccagaaagagtactccatcggaactggctctaccaagcaagaggcaaagcaactggctgccaaacttgcttacctccagattctttccgag

SEQ ID NO: 10 - DNA, 1346 bp, Artificial Sequence
  Cetuximab antibody heavy chain, nucleic acid sequence
caggtgcagcttaagcagtctggaccaggacttgttcagccttctcagtccctctccattacttgcactgtgtccggattctccctcaccaattacggtgttcactgggtgagacagtctccaggtaagggtcttgaatggctcggagtgatttggtccggtggcaacactgattacaacaccccattcacctccaggctctccatcaacaaggacaactccaagagccaggtgttcttcaagatgaactccctccagtccaacgacaccgctatctattattgcgctagggctctcacctactacgactacgagtttgcttactggggacagggaactctcgttactgtttccgctgcttctaccaagggaccatctgtttttccactcgctcccagctctaagtccacttctggtggaactgctgctcttggatgcctcgtgaaggattactttccagagccagtgaccgtgtcctggaactctggtgctcttacttcaggcgttcacactttcccagctgtgcttcaatcttccggactctactctctctcctctgttgtgactgtgccctcttcttcactcggcactcaaacctacatctgcaacgtgaaccacaagccatccaacaccaaggtggacaagaaggtcgagccaaagtcctgcgataagactcatacttgcccaccatgtccagctccagaacttcttggtggtccatccgttttcttgttcccaccaaagccaaaggacaccctcatgatctctaggactccagaggttacatgcgtggtggttgatgtgtctcatgaagatcctgaggtgaagttcaactggtacgttgacggtgttgaggtgcacaacgctaagactaagccacgtgaggaacagtacaactccacctacagggttgtgtctgtgcttactgtgttgcaccaggattggctcaacggcaaagagtacaagtgcaaggtgtccaacaaggctctccctgctccaatcgaaaagaccatctctaaggctaagggccagccaagagagccacaggtttacactcttccaccatccagggacgagctgaccaagaatcaggtttcccttacttgcctggtgaagggcttctacccatccgatattgctgttgagtgggagtctaatggccagcctgagaacaactacaagactactccaccagtgctcgactccgatggctcattcttcttgtactccaagctcaccgtggacaagtctagatggcagcagggaaacgtgttcagctgctctgttatgcatgaggcccttcacaaccactacacccagaagtccttgtctttgtctccaggcaa

SEQ ID NO: 11 - DNA, 642 bp, Artificial Sequence
  Cetuximab antibody light chain nucleic acid sequence
gacatcctgctcactcagtctccagtgatcctttctgtttccccaggtgagagggtgtcattttcttgcagggcttcccagtccatcggcactaacattcattggtatcagcagaggaccaacggctctccaaggctccttattaagtacgcctccgagtccatctccggcattccatctagattctccggatctggctccggcactgatttcaccctttccatcaactccgttgagtccgaggatatcgctgactactactgccagcagaacaacaactggccaactactttcggagctggcaccaagttggagcttaagagaactgttgctgccccatccgtgttcatcttcccaccatctgatgagcagctcaagtccggaactgcttctgttgtgtgcctcctcaacaacttctacccaagggaagctaaggtgcagtggaaggttgacaatgctctccagtccggaaactcccaagagtcagttacagagcaggactccaaggactctacctacagcctcagctctactctcactctcagcaaggctgattacgagaagcacaaggtgtacgcttgcgaggttacacaccagggactttcttcaccagtgaccaagtctttcaacaggggagagtgt

SEQ ID NO: 12 - DNA, 1016 bp, Artificial Sequence
  Plant promoter from a rubisco small subunit
aattcgatatcacgcttagacaaacaccccttgttatacaaagaatttcgctttacaaaatcaaattcgagaaaataatatatgcactaaataagatcattccgatccaatctaaccaattacgatacgctttgggtacacttgatttttgtttcagtagttacatatatcttgttttatatgctatctttaaggatcttcactcaaagactatttgttgatgttcttgatggggctcggaagatttgatatgatacactctaatctttaggagataccagccaggattatattcagtaagacaatcaaattttacgtgttcaaactcgttatcttttcatttaatggatgagccagaatctctatagaatgattgcaatcgagaatatgttcggccgatatccctttgttggcttcaatattctacatatcacacaagaatcgaccgtattgtaccctctttccataaaggaacacacagtatgcagatgcttttttcccacatgcagtaacataggtattcaaaaatggctaaaagaagttggataacaaattgacaactatttccatttctgttatataaatttcacaacacacaaaagcccgtaatcaagagtctgcccatgtacgaaataacttctattatttggtattgggcctaagcccagctcagagtacgtgggggtaccacatataggaaggtaacaaaatactgcaagatagccccataacgtaccagcctctccttaccacgaagagataagatataagacccaccctgccacgtgtcacatcgtcatggtggttaatgataagggattacatccttctatgtttgtggacatgatgcatgtaatgtcatgagccacatgatccaatggccacaggaacgtaagaatgtagatagatttgattttgtccgttagatagcaaacaacattataaaaggtgtgtatcaatacgaactaattcactcattggattcatagaagtccattcctcctaagtatctaaac

SEQ ID NO: 13 - DNA, 960 bp, Artificial Sequence
  Plant terminator from a rubisco small subunit
ggccgcataagttttactatttaccaagacttttgaatattaaccttcttgtaacgagtcggttaaatttgattgtttagggttttgtattatttttttttggtcttttaattcatcactttaattccctaattgtctgttcatttcgttgtttgtttccggatcgataatgaaatgtaagagatatcatatataaataataaattgtcgtttcatatttgcaatctttttttacaaacctttaattaattgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttcttttctcattaagcgaatcactacctaacacgtcattttgtgaaatattttttgaatgtttttatatagttgtagcattcctcttttcaaattagggtttgtttgagatagcatttcagccggttcatacaacttaaaagcatactctaatgctggaaaaaagactaaaaaatcttgtaagttagcgcagaatattgacccaaattatatacacacatgaccccatatagagactaattacacttttaaccactaataattattactgtattataacatctactaattaaacttgtgagtttttgctagaattattatcatatatactaaaaggcaggaacgcaaacattgccccggtactgtagcaactacggtagacgcattaattgtctatagtggacgcattaattaaccaaaaccgcctctttccccttcttcttgaagctggagctcg

SEQ ID NO: 14 - DNA, 117 bp, Gossypium sp.
atggctaggaagtcccttattttcccagtgatccttctcgccgtgctccttttttctccaccaatctactctgctggccacgattacagggatgctctccgtaaatcttccatggct

SEQ ID NO: 15 - DNA, 75 bp, Nicotiana sp.
atggccgctaggaagtcccttattttcccagtgattctcctcgccgtgctccttttttctccaccaatctactcc

SEQ ID NO: 16 - protein, 39 aa, Gossypium sp.
MARKSLIFPVILLAVLLFSPPIYSAGHDYRDALRKSSMA

SEQ ID NO: 17 - protein, 25 aa, Gossypium sp.
MAARKSLIFPVILLAVLLFSPPIYS

SEQ ID NO: 18 - DNA, 845 bp, Artificial Sequence
  CHN 50 S/M II
aggtacagaacgtgggaatctaagtttctgactctacatttctacatatttttaacttctaactctgaaagctcttattatactaaattgtgtaattccttagtaatatgtaaatttacttgaacttcttccagaaccactcccccaacctaattataactttctagctaaactcagcgatttttttggttcatcgtaagacattgtcagtcgaaatattgtactatatccatgtgaggctgattctttttaggaggaggacctaactcactcaagagacgccgggtgtaaccaggctctgttttttcgccaaaacaaaaaaactgggaatcaaactttcgtgctgcacgtagatattcgccatctttaagattaaattgaaaaccttctccttttttatgaaattcgtacttaaattttaaaaactcgcttgggcgtcattctgggtgaaattctttcttcctctgactaaattacaatttttttctaagtagaatgcgtgttcaaacatcaattcgaactcaaaaaattacttttctacatagtttaagaatttcttcagttcaagtaactagatctacatccaaacactcaccaagtgggcgcttggctataagaattgcaagatccccaaaaagtaatagaaactcttttcgtcgggtaatgatatttagaaactagaattatgcctcgacatgaacatgattttaggctgtttttaatcgtttgtctctaacctaaacgaaaactttgaaaagtagctctttggagtttttcaaaattttaagaaattcccaaaatacattttcaagtagaagttaaaaatactctaaaccaatgtcgat

SEQ ID NO: 19 - DNA, 1193 bp, Artificial Sequence
  TM6
taatatttagaaatttaattaacataaccaaggatttttatatcggtaataactctaatatggtatccaaatcagtctagaactctcttacctctaataagtaaaagtacttctaataaattcatatactttttctctcttctccgatctctctttgctcttctttttatgtatcctttcctttctaatagccttttatgagaagtaaacttttagggttggccccccctccccccacaattatatagtttcttactcagttgttggaatataattcaaattcttaaataattgacggtgacattgagttttactttgtggaagagaattagattctcgtgttagtaaaatcggttagtaattgatgatgcattatttttactctataatagagatgcaattttatttttgcattttgggatcaaattgtaatgcagtcatatattgatttcataaatgtttgggatattgttggttatttaactagaaatagacttcttatttcatatttattgttaaaatcctttattggagatgaattatttgttcaccgattagaagttgatagtcgcttttgttttagaagaaattttaccgtagaccaagttaaggagttttagaagcactttgcatgggagcattagtgtatgttatggctttatcaaatataggttttgaagattcagagagccaagaaaagctagaacccaagaactaggaagttagagtaattcacaataccataacgtgatataaaactttttattgtaactcaaatcggtaatattttttgctttagtcttaatcgataaattatttttttatattgattagttataggaggctcacaaagttgggaataattaaaatatcatattttgtatttgaacaatttatgaaatagtaattggtaaaaaatcactttaaatttttatcctatatccagaaggattatggtgtctggcatagttgtttggaagatttgaatcagggtaaaagtatgttgtaatttttattttgttataggcattttttgtgcttgattgttttgttgtcattatattttattatttggaagtgtatatatatgtttgattaaaatatagataatcaattttataagaaatttgcaacaattacacaaggataaagtctacaatatgcgagtaaaatttgattgaacctaggatgtc

SEQ ID NO: 20 - DNA, 1168 bp, Artificial Sequence
  Rb7
tcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 21 - DNA, 2393 bp, Artificial Sequence
  Marker cassette
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgaaatggcctcctccgagaacgtgatcactgagttcatgaggttcaaggtgaggatggaaggtactgtgaacggacacgagttcgagatcgaaggtgaaggtgagggtagaccatacgagggacacaacactgtgaagctcaaggtgacaaaaggtggcccacttccattcgcttgggatatcctttcaccacagttccagtacggctccaaggtttacgttaagcacccagctgatatccccgactacaagaagttgtctttcccagagggattcaagtgggagcgtgtgatgaatttcgaggatggtggtgtggctactgtgacccaggattcttcacttcaggatggctgcttcatctacaaggtgaagttcatcggggtgaacttcccatctgatggcccagtgatgcagaaaaagactatgggatgggaagcctccactgagaggctttatccaagagatggtgtgctcaagggcgagactcacaaggctcttaagctcaaagatggtggccactacctcgtcgagttcaagtctatctacatggccaagaagccagttcagctccccggttactactacgttgacgctaagctcgacatcaccagccacaacgaggattacactatcgtcgagcagtacgagaggactgaaggtaggcatcacttgttcctctgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtgaggtggcacttttcggggaaatgtgcgcggaacccctatttgtttatttttctaaatacagaattgtcgacttgtgtcgacaggtacagaacgtgggaatctaagtttctgactctacatttctacatatttttaacttctaactctgaaagctcttattatactaaattgtgtaattccttagtaatatgtaaatttacttgaacttcttccagaaccactcccccaacctaattataactttctagctaaactcagcgatttttttggttcatcgtaagacattgtcagtcgaaatattgtactatatccatgtgaggctgattctttttaggaggaggacctaactcactcaagagacgccgggtgtaaccaggctctgttttttcgccaaaacaaaaaaactgggaatcaaactttcgtgctgcacgtagatattcgccatctttaagattaaattgaaaaccttctccttttttatgaaattcgtacttaaattttaaaaactcgcttgggcgtcattctgggtgaaattctttcttcctctgactaaattacaatttttttctaagtagaatgcgtgttcaaacatcaattcgaactcaaaaaattacttttctacatagtttaagaatttcttcagttcaagtaactagatctacatccaaacactcaccaagtgggcgcttggctataagaattgcaagatccccaaaaagtaatagaaactcttttcgtcgggtaatgatatttagaaactagaattatgcctcgacatgaacatgattttaggctgtttttaatcgtttgtctctaacctaaacgaaaactttgaaaagtagctctttggagtttttcaaaattttaagaaattcccaaaatacattttcaagtagaagttaaaaatactctaaaccaatgtcgat

SEQ ID NO: 22 - DNA, 504 bp, Artificial Sequence
  artificial dsRBD
atggctggtgatctttccgctggcttcttcatggaagagttgaacacctacaggcaaaagcagggcgttgtgctcaagtaccaagagcttccaaattccggaccaccacacgataggcgtttcactttccaggtgatcatcgatggacgtgagttcccagaaggtgagggcagatctaagaaagaggctaagaacgctgctgctaagctcgctgttgagatcctgaacaaagagaagaaggccgtcagtccactcctcctgactactactaattcctccgagggactctccatgggaaactacattggactcatcaacaggatcgcccagaagaagaggctcaccgttaactacgagcaagtggcttctggtgttcatggaccagagggattccactacaaggtgaagatgggccagaaagagtactccatcggaactggctctaccaagcaagaggcaaagcaactggctgccaaacttgcttacctccagattctttccgag

SEQ ID NO: 23 - DNA, 51 bp, Artificial Sequence
  Gly linker heavy chain
gggcccggtggaggaggctctggtggaggcggtagcggaggcggagggtct

SEQ ID NO: 24 - DNA, 45 bp, Artificial Sequence
  Gly linker light chain
ggtggaggaggctctggtggaggcggtagcggaggcggagggtct

SEQ ID NO: 25 - DNA, 1473 bp, Artificial Sequence
  Nucleic acid sequence of Cetuximab Heavy Chain with apoplast signal peptide
atggctaggaagtcccttattttcccagtgatccttctcgccgtgctccttttttctccaccaatctactctgctggccacgattacagggatgctctccgtaaatcttccatggctcaggtgcagcttaagcagtctggaccaggacttgttcagccttctcagtccctctccattacttgcactgtgtccggattctccctcaccaattacggtgttcactgggtgagacagtctccaggtaagggtcttgaatggctcggagtgatttggtccggtggcaacactgattacaacaccccattcacctccaggctctccatcaacaaggacaactccaagagccaggtgttcttcaagatgaactccctccagtccaacgacaccgctatctattattgcgctagggctctcacctactacgactacgagtttgcttactggggacagggaactctcgttactgtttccgctgcttctaccaagggaccatctgtttttccactcgctcccagctctaagtccacttctggtggaactgctgctcttggatgcctcgtgaaggattactttccagagccagtgaccgtgtcctggaactctggtgctcttacttcaggcgttcacactttcccagctgtgcttcaatcttccggactctactctctctcctctgttgtgactgtgccctcttcttcactcggcactcaaacctacatctgcaacgtgaaccacaagccatccaacaccaaggtggacaagaaggtcgagccaaagtcctgcgataagactcatacttgcccaccatgtccagctccagaacttcttggtggtccatccgttttcttgttcccaccaaagccaaaggacaccctcatgatctctaggactccagaggttacatgcgtggtggttgatgtgtctcatgaagatcctgaggtgaagttcaactggtacgttgacggtgttgaggtgcacaacgctaagactaagccacgtgaggaacagtacaactccacctacagggttgtgtctgtgcttactgtgttgcaccaggattggctcaacggcaaagagtacaagtgcaaggtgtccaacaaggctctccctgctccaatcgaaaagaccatctctaaggctaagggccagccaagagagccacaggtttacactcttccaccatccagggacgagctgaccaagaatcaggtttcccttacttgcctggtgaagggcttctacccatccgatattgctgttgagtgggagtctaatggccagcctgagaacaactacaagactactccaccagtgctcgactccgatggctcattcttcttgtactccaagctcaccgtggacaagtctagatggcagcagggaaacgtgttcagctgctctgttatgcatgaggcccttcacaaccactacacccagaagtccttgtctttgtctccaggcaaggggccctaa

SEQ ID NO: 26 - DNA, 717 bp, Artificial Sequence
  Nucleic acid sequence of Cetuximab Light Chain with apoplast signal peptide
atggccgctaggaagtcccttattttcccagtgattctcctcgccgtgctccttttttctccaccaatctactccgacatcctgctcactcagtctccagtgatcctttctgtttccccaggtgagagggtgtcattttcttgcagggcttcccagtccatcggcactaacattcattggtatcagcagaggaccaacggctctccaaggctccttattaagtacgcctccgagtccatctccggcattccatctagattctccggatctggctccggcactgatttcaccctttccatcaactccgttgagtccgaggatatcgctgactactactgccagcagaacaacaactggccaactactttcggagctggcaccaagttggagcttaagagaactgttgctgccccatccgtgttcatcttcccaccatctgatgagcagctcaagtccggaactgcttctgttgtgtgcctcctcaacaacttctacccaagggaagctaaggtgcagtggaaggttgacaatgctctccagtccggaaactcccaagagtcagttacagagcaggactccaaggactctacctacagcctcagctctactctcactctcagcaaggctgattacgagaagcacaaggtgtacgcttgcgaggttacacaccagggactttcttcaccagtgaccaagtctttcaacaggggagagtgt

SEQ ID NO: 27 - DNA, 1272 bp, Artificial Sequence
  Nucleic acid sequence of dsRBD-linker- Cetuximab Light Chain with apoplast signal peptide
atggccgctaggaagtcccttattttcccagtgattctcctcgccgtgctccttttttctccaccaatctactccatgatggctggtgatctttccgctggcttcttcatggaagagttgaacacctacaggcaaaagcagggcgttgtgctcaagtaccaagagcttccaaattccggaccaccacacgataggcgtttcactttccaggtgatcatcgatggacgtgagttcccagaaggtgagggcagatctaagaaagaggctaagaacgctgctgctaagctcgctgttgagatcctgaacaaagagaagaaggccgtcagtccactcctcctgactactactaattcctccgagggactctccatgggaaactacattggactcatcaacaggatcgcccagaagaagaggctcaccgttaactacgagcaagtggcttctggtgttcatggaccagagggattccactacaaggtgaagatgggccagaaagagtactccatcggaactggctctaccaagcaagaggcaaagcaactggctgccaaacttgcttacctccagattctttccgagggtggaggaggctctggtggaggcggtagcggaggcggagggtctgacatcctgctcactcagtctccagtgatcctttctgtttccccaggtgagagggtgtcattttcttgcagggcttcccagtccatcggcactaacattcattggtatcagcagaggaccaacggctctccaaggctccttattaagtacgcctccgagtccatctccggcattccatctagattctccggatctggctccggcactgatttcaccctttccatcaactccgttgagtccgaggatatcgctgactactactgccagcagaacaacaactggccaactactttcggagctggcaccaagttggagcttaagagaactgttgctgccccatccgtgttcatcttcccaccatctgatgagcagctcaagtccggaactgcttctgttgtgtgcctcctcaacaacttctacccaagggaagctaaggtgcagtggaaggttgacaatgctctccagtccggaaactcccaagagtcagttacagagcaggactccaaggactctacctacagcctcagctctactctcactctcagcaaggctgattacgagaagcacaaggtgtacgcttgcgaggttacacaccagggactttcttcaccagtgaccaagtctttcaacaggggagagtgttga

SEQ ID NO: 28 - DNA, 2025 bp, Artificial Sequence
  Nucleic acid sequence of Cetuximab Heavy Chain with apoplast signal peptide - linker - dsRBD
atggctaggaagtcccttattttcccagtgatccttctcgccgtgctccttttttctccaccaatctactctgctggccacgattacagggatgctctccgtaaatcttccatggctcaggtgcagcttaagcagtctggaccaggacttgttcagccttctcagtccctctccattacttgcactgtgtccggattctccctcaccaattacggtgttcactgggtgagacagtctccaggtaagggtcttgaatggctcggagtgatttggtccggtggcaacactgattacaacaccccattcacctccaggctctccatcaacaaggacaactccaagagccaggtgttcttcaagatgaactccctccagtccaacgacaccgctatctattattgcgctagggctctcacctactacgactacgagtttgcttactggggacagggaactctcgttactgtttccgctgcttctaccaagggaccatctgtttttccactcgctcccagctctaagtccacttctggtggaactgctgctcttggatgcctcgtgaaggattactttccagagccagtgaccgtgtcctggaactctggtgctcttacttcaggcgttcacactttcccagctgtgcttcaatcttccggactctactctctctcctctgttgtgactgtgccctcttcttcactcggcactcaaacctacatctgcaacgtgaaccacaagccatccaacaccaaggtggacaagaaggtcgagccaaagtcctgcgataagactcatacttgcccaccatgtccagctccagaacttcttggtggtccatccgttttcttgttcccaccaaagccaaaggacaccctcatgatctctaggactccagaggttacatgcgtggtggttgatgtgtctcatgaagatcctgaggtgaagttcaactggtacgttgacggtgttgaggtgcacaacgctaagactaagccacgtgaggaacagtacaactccacctacagggttgtgtctgtgcttactgtgttgcaccaggattggctcaacggcaaagagtacaagtgcaaggtgtccaacaaggctctccctgctccaatcgaaaagaccatctctaaggctaagggccagccaagagagccacaggtttacactcttccaccatccagggacgagctgaccaagaatcaggtttcccttacttgcctggtgaagggcttctacccatccgatattgctgttgagtgggagtctaatggccagcctgagaacaactacaagactactccaccagtgctcgactccgatggctcattcttcttgtactccaagctcaccgtggacaagtctagatggcagcagggaaacgtgttcagctgctctgttatgcatgaggcccttcacaaccactacacccagaagtccttgtctttgtctccaggcaaggggcccggtggaggaggctctggtggaggcggtagcggaggcggagggtctatgatggctggtgatctttccgctggcttcttcatggaagagttgaacacctacaggcaaaagcagggcgttgtgctcaagtaccaagagcttccaaattccggaccaccacacgataggcgtttcactttccaggtgatcatcgatggacgtgagttcccagaaggtgagggcagatctaagaaagaggctaagaacgctgctgctaagctcgctgttgagatcctgaacaaagagaagaaggccgtcagtccactcctcctgactactactaattcctccgagggactctccatgggaaactacattggactcatcaacaggatcgcccagaagaagaggctcaccgttaactacgagcaagtggcttctggtgttcatggaccagagggattccactacaaggtgaagatgggccagaaagagtactccatcggaactggctctaccaagcaagaggcaaagcaactggctgccaaacttgcttacctccagattctttccgagtga

(19) United States
(12) Patent Application Publication
(10) Pub. No.: US 2020/0407453 A1
(43) Pub. Date: Dec. 31, 2020

(54) A RECOMBINANT PROTEIN COMPRISING A DOUBLE STRANDED RNA BINDING DOMAIN

(71) Applicant: TARGIMMUNE THERAPEUTICS AG, Basel (CH)

(72) Inventors: Alexander LEVITZKI, Jerusalem (IL); Lital FRIEDMAN, Hashmonaim (IL); Shoshana KLEIN, Maale Adumim (IL)

(21) Appl. No.: 16/978,976

(22) PCT Filed: Mar. 8, 2019
(86) PCT No.: PCT/EP2019/055912

§ 371 Date: Sep. 8, 2020

(30) Foreign Application Priority Data
Mar. 8, 2018 (EP) 18160819.1

(51) Int. Cl. C07K 16/28; C07K 14/47; A61K 38/17; A61K 39/395; A61K 31/7088; A61P 35/00

(52) U.S. Cl. CPC C07K 16/2863; C07K 14/4702; A61K 38/1709; A61K 39/39558; A61K 39/3955; C07K 2319/33; A61P 35/00; C07K 2319/30; C07K 2317/24; C07K 2319/85; A61K 31/7088

(57) ABSTRACT

The present invention relates to a recombinant protein with a nucleic acid binding domain. In particular, the present invention relates to a recombinant protein comprising a double stranded RNA (dsRNA) binding domain and a Cetuximab antibody; a complex further comprising dsRNA; a nucleic acid sequence; and a pharmaceutical composition comprising the recombinant protein or the complex of the invention and a pharmaceutically acceptable carrier. Further, the present invention relates to the recombinant protein, complex or pharmaceutical composition of the invention for use in the treatment of cancer, wherein said cancer is characterized by EGFR-overexpressing cells.

The present invention relates to the field of recombinant proteins with nucleic acid binding domains. In particular, the present invention relates to a recombinant or chimeric protein comprising a double stranded RNA (dsRNA) binding domain and a Cetuximab antibody; a complex further comprising dsRNA; a nucleic acid sequence; and a pharmaceutical composition comprising the recombinant protein or the complex of the invention. Further, the present invention relates to the recombinant protein, complex or pharmaceutical composition of the invention for use in the treatment of cancer, wherein said cancer is characterized by EGFR-overexpressing cells.

### RELATED ART

Cancer such as colorectal or lung cancers is still a devastating disease that has baffled researchers over the years. Elevated levels of the EGFR (erbB-1) and its cognate ligands have been identified as a common component of numerous cancer types. A study by Nicholson et al. showed several cancer types including head and neck, ovarian, cervical, bladder, oesophageal, gastric, breast, endometrial, colorectal cancers, and non-small cell lung cancer (NSCLC) express elevated levels of EGFR relative to normal tissues and have been studied in sufficient depth to allow sound judgements to be made concerning the association between EGFR and patient outlook (Nicholson et al., “EGFR and cancer prognosis”, Eur. J. Cancer, vol. 37, pp. 9-15, 2001).

Several strategies aimed at blocking the erbB receptors and their signaling pathways are currently undergoing preclinical and clinical investigation. Among these are immunotherapy with monoclonal and bispecific antibodies that bind to erbB receptors or their ligands; antibody/immunotoxin conjugates that selectively target cancer cells; small-molecule therapy with tyrosine kinase inhibitors that block phosphorylation of erbB receptors; antisense oligonucleotides that reduce transcription of erbB receptor genes; ribozymes that degrade erbB mRNA; and ansamycin analogs that promote degradation of certain members of the erbB family through the ubiquitin pathway. Several MAb drugs are undergoing clinical investigation, including Trastuzumab, a humanized MAb; Cetuximab (C225), a chimeric MAb; and MDXH210, a humanized bispecific antibody; and MDX447, a bispecific antibody (Slichenmyer et al., Anticancer Therapy Targeting the ErbB Family of Receptor Tyrosine Kinases, Seminars in Oncology, vol. 28 (5), Suppl 16, pp. 67-79, 2001).

Cetuximab (IMC-C225, Erbitux®) is a recombinant, human/mouse chimeric IgG1 monoclonal antibody that binds specifically to the epidermal growth factor receptor (EGFR, HER1, c-ErbB-1) on both normal and tumor cells and competitively inhibits the binding of epidermal growth factor (EGF) and other ligands, such as transforming growth factor-alpha. Binding of Cetuximab to the EGFR blocks phosphorylation and activation of receptor-associated kinases, resulting in inhibition of cell growth, induction of apoptosis, and decreased matrix metalloproteinase and vascular endothelial growth factor production. EGFR is constitutively expressed in many normal epithelial tissues, including the skin and hair follicles. Over-expression of EGFR is also detected in many human cancers including those of the colon and rectum (NIH, National Cancer Institute, FDA Approval for Cetuximab, Jul. 2, 2013).

Cetuximab is approved for use in the treatment of EGFR-expressing, metastatic or recurrent colorectal carcinoma, locally or regionally advanced squamous cell carcinoma of the head and neck (SCCHN) and for use in combination with FOLFIRI (irinotecan, 5-fluorouracil, and leucovorin) for first-line treatment of patients with K-ras mutation-negative (wild-type), EGFR-expressing metastatic colorectal cancer (mCRC)(NIH, National Cancer Institute, FDA Approval for Cetuximab, Jul. 2, 2013).

The prognosis of patients with metastatic colorectal cancer remains poor despite the impressive improvement of treatments observed over the last years. Cetuximab and Panitumumab are effective in terms of progression-free survival, overall survival, response rate, and quality of life observed in several phase III clinical trials among different lines of treatment. However, they were shown only to be effective in a subset of patients, and even the responders eventually become resistant by developing secondary resistance. There are a number of suggested molecular mechanisms that underlie both primary and acquired resistance to anti-EGFR drugs. The most frequent mechanisms of resistance are a result of genomic alterations in downstream effectors (e.g., KRAS, NRAS, BRAF, and PIK3CA) of the EGFR signaling pathway (Sforza et al., Mechanisms of resistance to anti-epidermal growth factor receptor inhibitors in metastatic colorectal cancer, World J Gastroenterol, 2016, vol. 22(28), pp. 6345-6361).

Viral double-stranded RNA (dsRNA) mimetics have been explored in cancer immunotherapy to promote antitumoral immune response. Polyinosinic-polycytidylic acid (polyIC) and polyadenylic-polyuridylic acid (polyAU) are synthetic analogs of viral dsRNA and strong inducers of type I interferon (IFN-I). A direct effect of synthetic dsRNA on cancer cells has been demonstrated, based on induction of IFN-I production, which in turn promotes the apoptosis of cancer cells via an autocrine signaling loop (Gatti et al., Direct effect of dsRNA mimetics on cancer cells induces endogenous IFN-β production capable of improving dendritic cell function, Eur. J. Immunol. 2013, vol. 43, pp. 1849-1861).

PolyIC complexes are effective IFN inducers in humans, but their toxicity limits their use in cancer patients (Krown et al., Phase I trials of poly(I,C) complexes in advanced cancer J. Biol. Response Mod. 1985, vol. 4(6), pp. 640-649). In order to specifically introduce polyIC into EGFR overexpressing cells, complexes of polyethylenimine (25 kDa), polyethylene-glycol and mouse EGF have been utilized (Shir et al., EGF receptor-targeted synthetic double-stranded RNA eliminates glioblastoma, breast cancer, and adenocarcinoma tumors in mice, PLoS Med. 2006, vol. 3(1), p. e6).

However, there is still a high need for efficient and well-tolerated cancer therapeutics and carriers that are capable to selectively deliver RNA to tumor cells

### SUMMARY OF THE INVENTION

In a first aspect, the invention relates to a recombinant protein comprising a double stranded RNA (dsRNA) binding domain and a Cetuximab antibody.

In a second aspect, the invention relates to a complex comprising the recombinant protein of the invention and dsRNA.

In a further aspect, the invention relates to a pharmaceutical composition comprising the recombinant protein or the complex of the invention and a pharmaceutically acceptable carrier.

In an additional aspect, the invention relates to the recombinant protein, complex or pharmaceutical composition of the invention for use in the treatment of cancer, wherein said cancer is characterized by EGFR-overexpressing cells.

In a further aspect, the invention relates to a nucleic acid sequence comprising a nucleic acid sequence encoding the recombinant protein of the invention.

The inventors developed a recombinant protein comprising a Cetuximab antibody bound to a double-stranded RNA binding domain (dsRBD). The recombinant protein of the invention is capable of binding EGFR via the Cetuximab antibody, and thus mediates antibody-dependent cellular effects, such as Fc-mediated effects. The Cetuximab antibody binds to the extracellular domain, thus inhibiting EGFR downstream signaling, inducing receptor internalization and downregulation as well as mediating antibody-dependent cellular cytotoxicity (ADCC). However, this is not the only effect of Cetuximab in the recombinant protein of the invention.

The recombinant protein of the invention is able to selectively deliver dsRNA, such as polyIC, to tumor cells over-expressing EGFR, thus inducing anti-cancer effects via dsRNA, such as cytotoxicity, apoptosis and recruitment of immune cells as well as a so-called “bystander effect”, i.e. nearby cancer cells that do not express EGFR are killed from the effects of the targeted dsRNA.

Cetuximab antibody included in the recombinant protein targets EGFR and thus exploits the over-expression of EGFR on cancer cells to selectively deliver cytotoxic dsRNA molecules to cancer cells and to induce selective uptake of dsRNA into these cells. Thereby, side effects are reduced, and an immune response is induced selectively in cancer cells.

Moreover, the response to Cetuximab antibody will be enhanced by dsRNA, such as polyIC which activates TLR3. Thus, dsRNA, such as polyIC provides an effective mean to synergistically improve the efficacy of Cetuximab antibody-based anticancer regimen.

Further, the recombinant protein of the invention is advantageous over a chemical vector as it is precisely defined and can be simply produced at low cost.

### DESCRIPTION OF THE FIGURES

FIG. 1: Schematic design of (A) Cetuximab, (B) Cetuximab-LC-dsRBD (dsRBD attached to the N terminus of the light chain (LC) of Cetuximab), (C) Cetuximab-HC-dsRBD (dsRBD attached to the C terminus of the heavy chain (HC) of Cetuximab) FIG. 2: Design of Cetuximab-DsRed pUC57 vector comprising genes encoding the heavy chain and light chain of the Cetuximab antibody, and a gene expressing DsRed—a red fluorescent protein used as a screenable marker. These genes were codon-optimized for tobacco. The heavy and light chains are each flanked by the rubisco small subunit promoter and terminator and each contain a signal peptide directing them to the apoplast, the space outside of the plant cell plasma membrane. The DsRed gene is flanked by the CaMV 35S promoter and terminator. Surrounding each expression unit are matrix attachment regions (MARs)—labeled CHN S/M II, TM6 and Rb7 in the figure—which have been shown to enhance gene expression.

FIG. 3: Intermediate vector pUC57 encoding Cetuximab heavy and light chains and screenable marker DsRed after successful Golden Gate assembly.

FIG. 4: Cetuximab-light chain-dsRBD-DsRed in pUC57 (A); Cetuximab-heavy chain-dsRBD-DsRed in pUC57 (B).

FIG. 5: Map of the binary agrobacterium expression vector pBINPLUS for plant transformation.

FIG. 6: Western blot of selected Cetuximab expressing tobacco plants, using anti-human IgG HRP antibody. Bands at ˜50 and ˜25 kDa represent the heavy and light chains, respectively. 100 ng of commercial Cetuximab was run for comparison. A sample from wild type (WT) plant was used as control. Detailed Description of the Invention FIG. 7: Western blot of selected Cetuximab-LC-dsRBD expressing tobacco plants, using anti-human IgG HRP antibody (A) and mouse anti-human PKR followed by anti-mouse IgG antibody (B). Bands at ˜50 and ˜45 kDa represent the heavy chain and light chain-dsRBD, respectively.

FIG. 8: Western blot of selected Cetuximab-HCdsRBD expressing tobacco plants, using anti-human IgG HRP antibody (A) and mouse anti-human PKR followed by anti-mouse IgG antibody (B). Bands at ˜75 and ˜25 kDa represent the heavy chain-dsRBD and light chain, respectively.

FIG. 9: Western blots of Protein A bead purification results. For each purification attempt of Cetuximab (A), Cetuximab-LC-dsRBD (B) and Cetuximab-HC-dsRBD (C), samples were run from the lysate before purification, the unbound proteins, the bound proteins (beads before elution) and the eluted protein. Blots were detected using an anti-human IgG-HRP antibody. For the Cetuximab-dsRBD chimeras, mouse anti-PKR followed by anti-mouse-HRP antibody was also used for detection (right pane of B and C).

FIG. 10: Protein A purification chromatography of plant derived Cetuximab. The elution peak was observed between 87 and 90 ml (x-axis)(A). Coomassie staining of selected fraction samples; the protein is found in fractions 6-11 (B). Western blot analysis using anti-human IgG-HRP antibody shows Cetuximab heavy and light chains in lysate (before purification) as well as in eluted fractions, but not in unbound or wash fractions (C).

FIG. 11: Protein A purification chromatography of plant derived Cetuximab-LC-dsRBD. The elution peak was observed between 82 and 90 ml (x-axis) (A). Western blot analysis using anti-human IgG-HRP antibody shows elution of Cetuximab heavy chain, and a ˜25 kDa band that could be the original light chain, but not LC-dsRBD (B).

FIG. 12: Specific binding of commercial Cetuximab, tobacco-expressed Cetuximab and Cetuximab-LC-dsRBD to EGFR.

TABLE OF SEQUENCES

| SEQ ID NO: | Sequence |
|---|---|
| 1 | Cetuximab antibody heavy chain, amino acid sequence: |
| | QVQLKQSGPGLVQPSQSLSITCTVSGFSLTNYGVHWVRQSPGKGLEWLGVIWSGGNTDYNTPFTSRLSINKD |
| | NSKSQVFFKMNSLQSNDTAIYYCARALTYYDYEFAYWGQGTLVTVSAASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKLTVDKSRWQQGNVFSCSVMHE |
| | ALHNHYTQKSLSLSPGK |
| 2 | Cetuximab antibody light chain, amino acid sequence: |
| | DILLTQSPVILSVSPGERVSFSCRASQSIGTNIHWYQQRTNGSPRLLIKYASESISGIPSRFSGSGSGTDFT |
| | LSINSVESEDIADYYCQQNNNWPTTFGAGTKLELKRTVAAPSVFIFPPSDEQLKSGTASVVCLLNNFYPREA |
| | KVQWKVDNALQSGNSQESVTEQDSKDSTYSLSSTLTLSKADYEKHKVYACEVTHQGLSSPVTKSFNRGEC |
| 3 | Spacer 1 amino acid sequence: |
| | GGGGSGGGGSGGGGS |
| 4 | Spacer 2 amino acid sequence: |
| | GPGGGGSGGGGSGGGGS |
| 5 | dsRBm1 amino acid sequence: |
| | SAGFFMEELNTYRQKQGVVLKYQELPNSGPPHDRRFTFQVIIDGREFPEGEGRSKKEAKNAAAKLAVEILNK |
| | EK |
| 6 | dsRBm2 amino acid sequence: |
| | LSMGNYIGLINRIAQKKRLTVNYEQVASGVHGPEGFHYKVKMGQKEYSIGTGSTKQEAKQLAAKLAYLQILS |
| | E |
| 7 | Linker linking dsRBm1 and dsRBm2 amino acid sequence: |
| | KKAVSPLLLTTTNSSEGLSMG |
| 8 | dsRBD amino acid sequence: |
| | MAGDLSAGFFMEELNTYRQKQGVVLKYQELPNSGPPHDRRFTFQVIIDGREFPEGEGRSKKEAKNAAAKLAV |
| | EILNKEKKAVSPLLLTTTNSSEGLSMGNYIGLINRIAQKKRLTVNYEQVASGVHGPEGFHYKVKMGQKEYSI |
| | GTGSTKQEAKQLAAKLAYLQILSE |
| 9 | dsRBD nucleic acid sequence: |
| | ATGATGGCTGGTGATCTTTCCGCTGGCTTCTTCATGGAAGAGTTGAACACCTACAGGCAAAAGCAGGGCGTT |
| | GTGCTCAAGTACCAAGAGCTTCCAAATTCCGGACCACCACACGATAGGCGTTTCACTTTCCAGGTGATCATC |
| | GATGGACGTGAGTTCCCAGAAGGTGAGGGCAGATCTAAGAAAGAGGCTAAGAACGCTGCTGCTAAGCTCGCT |
| | GTTGAGATCCTGAACAAAGAGAAGAAGGCCGTCAGTCCACTCCTCCTGACTACTACTAATTCCTCCGAGGGA |
| | CTCTCCATGGGAAACTACATTGGACTCATCAACAGGATCGCCCAGAAGAAGAGGCTCACCGTTAACTACGAG |
| | CAAGTGGCTTCTGGTGTTCATGGACCAGAGGGATTCCACTACAAGGTGAAGATGGGCCAGAAAGAGTACTCC |
| | ATCGGAACTGGCTCTACCAAGCAAGAGGCAAAGCAACTGGCTGCCAAACTTGCTTACCTCCAGATTCTTTCC |
| | GAG |
| 10 | Cetuximab antibody heavy chain, nucleic acid sequence: |
| | CAGGTGCAGCTTAAGCAGTCTGGACCAGGACTTGTTCAGCCTTCTCAGTCCCTCTCCATTACTTGCACTGTG |
| | TCCGGATTCTCCCTCACCAATTACGGTGTTCACTGGGTGAGACAGTCTCCAGGTAAGGGTCTTGAATGGCTC |
| | GGAGTGATTTGGTCCGGTGGCAACACTGATTACAACACCCCATTCACCTCCAGGCTCTCCATCAACAAGGAC |
| | AACTCCAAGAGCCAGGTGTTCTTCAAGATGAACTCCCTCCAGTCCAACGACACCGCTATCTATTATTGCGCT |
| | AGGGCTCTCACCTACTACGACTACGAGTTTGCTTACTGGGGACAGGGAACTCTCGTTACTGTTTCCGCTGCT |
| | TCTACCAAGGGACCATCTGTTTTTCCACTCGCTCCCAGCTCTAAGTCCACTTCTGGTGGAACTGCTGCTCTT |
| | GGATGCCTCGTGAAGGATTACTTTCCAGAGCCAGTGACCGTGTCCTGGAACTCTGGTGCTCTTACTTCAGGC |
| | GTTCACACTTTCCCAGCTGTGCTTCAATCTTCCGGACTCTACTCTCTCTCCTCTGTTGTGACTGTGCCCTCT |
| | TCTTCACTCGGCACTCAAACCTACATCTGCAACGTGAACCACAAGCCATCCAACACCAAGGTGGACAAGAAG |
| | GTCGAGCCAAAGTCCTGCGATAAGACTCATACTTGCCCACCATGTCCAGCTCCAGAACTTCTTGGTGGTCCA |
| | TCCGTTTTCTTGTTCCCACCAAAGCCAAAGGACACCCTCATGATCTCTAGGACTCCAGAGGTTACATGCGTG |
| | GTGGTTGATGTGTCTCATGAAGATCCTGAGGTGAAGTTCAACTGGTACGTTGACGGTGTTGAGGTGCACAAC |
| | GCTAAGACTAAGCCACGTGAGGAACAGTACAACTCCACCTACAGGGTTGTGTCTGTGCTTACTGTGTTGCAC |
| | CAGGATTGGCTCAACGGCAAAGAGTACAAGTGCAAGGTGTCCAACAAGGCTCTCCCTGCTCCAATCGAAAAG |
| | ACCATCTCTAAGGCTAAGGGCCAGCCAAGAGAGCCACAGGTTTACACTCTTCCACCATCCAGGGACGAGCTG |
| | ACCAAGAATCAGGTTTCCCTTACTTGCCTGGTGAAGGGCTTCTACCCATCCGATATTGCTGTTGAGTGGGAG |
| | TCTAATGGCCAGCCTGAGAACAACTACAAGACTACTCCACCAGTGCTCGACTCCGATGGCTCATTCTTCTTG |
| | TACTCCAAGCTCACCGTGGACAAGTCTAGATGGCAGCAGGGAAACGTGTTCAGCTGCTCTGTTATGCATGAG |
| | GCCCTTCACAACCACTACACCCAGAAGTCCTTGTCTTTGTCTCCAGGCAA |
| 11 | Cetuximab antibody light chain, nucleic acid sequence: |
| | GACATCCTGCTCACTCAGTCTCCAGTGATCCTTTCTGTTTCCCCAGGTGAGAGGGTGTCATTTTCTTGCAGG |
| | GCTTCCCAGTCCATCGGCACTAACATTCATTGGTATCAGCAGAGGACCAACGGCTCTCCAAGGCTCCTTATT |
| | AAGTACGCCTCCGAGTCCATCTCCGGCATTCCATCTAGATTCTCCGGATCTGGCTCCGGCACTGATTTCACC |
| | CTTTCCATCAACTCCGTTGAGTCCGAGGATATCGCTGACTACTACTGCCAGCAGAACAACAACTGGCCAACT |
| | ACTTTCGGAGCTGGCACCAAGTTGGAGCTTAAGAGAACTGTTGCTGCCCCATCCGTGTTCATCTTCCCACCA |
| | TCTGATGAGCAGCTCAAGTCCGGAACTGCTTCTGTTGTGTGCCTCCTCAACAACTTCTACCCAAGGGAAGCT |
| | AAGGTGCAGTGGAAGGTTGACAATGCTCTCCAGTCCGGAAACTCCCAAGAGTCAGTTACAGAGCAGGACTCC |
| | AAGGACTCTACCTACAGCCTCAGCTCTACTCTCACTCTCAGCAAGGCTGATTACGAGAAGCACAAGGTGTAC |
| | GCTTGCGAGGTTACACACCAGGGACTTTCTTCACCAGTGACCAAGTCTTTCAACAGGGGAGAGTGT |
| 12 | Nucleic acid sequence of a plant promoter from a rubisco small subunit |
| 13 | Nucleic acid sequence of a plant terminator from a rubisco small subunit |
| 14 | Nucleic acid sequence encoding a cotton apoplast signal peptide |
| 15 | Nucleic acid sequence of a tobacco apoplast signal peptide |
| 16 | Amino acid sequence encoding a cotton apoplast signal peptide |
| 17 | Amino acid sequence of the tobacco apoplast signal peptide |
| 18 | Nucleic acid sequence of CHN 50 S/M II |
| 19 | Nucleic acid sequence of TM6 |
| 20 | Nucleic acid sequence of Rb7 |
| 21 | Marker cassette |
| 22 | dsRBD nucleic acid sequence 2: |
| | ATGGCTGGTGATCTTTCCGCTGGCTTCTTCATGGAAGAGTTGAACACCTACAGGCAAAAGCAGGGCGTTGTG |
| | CTCAAGTACCAAGAGCTTCCAAATTCCGGACCACCACACGATAGGCGTTTCACTTTCCAGGTGATCATCGAT |
| | GGACGTGAGTTCCCAGAAGGTGAGGGCAGATCTAAGAAAGAGGCTAAGAACGCTGCTGCTAAGCTCGCTGTT |
| | GAGATCCTGAACAAAGAGAAGAAGGCCGTCAGTCCACTCCTCCTGACTACTACTAATTCCTCCGAGGGACTC |
| | TCCATGGGAAACTACATTGGACTCATCAACAGGATCGCCCAGAAGAAGAGGCTCACCGTTAACTACGAGCAA |
| | GTGGCTTCTGGTGTTCATGGACCAGAGGGATTCCACTACAAGGTGAAGATGGGCCAGAAAGAGTACTCCATC |
| | GGAACTGGCTCTACCAAGCAAGAGGCAAAGCAACTGGCTGCCAAACTTGCTTACCTCCAGATTCTTTCCGAG |
| 23 | Nucleic acid sequence of linker for heavy chain |
| | GGGCCCGGTGGAGGAGGCTCTGGTGGAGGCGGTAGCGGAGGCGGAGGGTCT |
| 24 | Nucleic acid sequence of linker for light chain |
| | GGTGGAGGAGGCTCTGGTGGAGGCGGTAGCGGAGGCGGAGGGTCT |
| 25 | Nucleic acid sequence of Cetuximab Heavy Chain with apoplast signal |
| | peptide |
| 26 | Nucleic acid sequence of Cetuximab Light Chain with apoplast signal |
| | peptide |
| 27 | Nucleic acid sequence of dsRBD-linker-Cetuximab Light Chain with |
| | apoplast signal peptide |
| 28 | Nucleic acid sequence of Cetuximab Heavy Chain with apoplast signal |
| | peptide-linker-dsRBD |

### DETAILED DESCRIPTION OF THE INVENTION

Throughout this specification and the claims which follow, unless the context requires otherwise, the word “comprise” or the word “include”, and variations such as “comprises/includes” and “comprising/including”, are to be understood to imply the inclusion of an element, stated integer, step or a group thereof but not the exclusion of any other element, stated integer, step or a group thereof.

As used in this specification and the appended claims, the singular forms “a”, “an”, and “the” include plural referents, unless the content clearly dictates otherwise. The term “about” when used in connection with a numerical value is meant to encompass numerical values within a range having a lower limit that is 0-10% smaller than the indicated numerical value and having an upper limit that is 0-10% larger than the indicated numerical value.

In a first aspect, the invention relates to recombinant protein comprising a double stranded RNA (dsRNA) binding domain and a Cetuximab antibody.

Cetuximab (Erbitux®) has an epidermal growth factor receptor binding Fab (fragment, antigen-binding) region. Cetuximab is composed of the Fv (variable; antigen-binding) regions of the 225 murine EGFR monoclonal antibody specific for the N-terminal portion of human EGFR with human IgG1 heavy and kappa light chain constant (framework) regions.

Cetuximab full antibody includes two heavy chains and two light chains. Cetuximab heavy chain and light chain sequences are known in the art. For instance, a Cetuximab heavy chain or light chain can have a Cetuximab heavy chain sequence or Cetuximab light chain sequence as disclosed in any of (1) Li et al., Structural basis for inhibition of the epidermal growth factor receptor by Cetuximab, Cancer Cell 2005, vol. 7, pp. 301-311; (2) Dubois et al., Immunopurification and Mass Spectrometric Quantification of the Active Form of a Chimeric Therapeutic Antibody in Human Serum, Anal. Chem 2008; vol. 80: pp. 1737-1745; (3) Cetuximab at HVIGT database, sequence available online at www.imgt.org/3Dstructure-DB/cgi/details.cgi?pdbcode=7906; (4) Ayoub et al., Correct primary structure assessment and extensive glyco-profiling of Cetuximab by a combination of intact, middle-up, middle-down and bottom-up ESI and MALDI mass spectrometry techniquesm, Abs 2013, vol. 5(5), pp. 699-710 (inclusive of supplemental material); or (5) Cetuximab at DrugBank (https://www.drugbank.ca/drugs/DB00002); each of which is hereby incorporated by reference in its entirety.

Most preferably, Cetuximab heavy chain (CTX-HC)
has the following sequence of SEQ ID NO 1:
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Most preferably, Cetuximab light chain (CTX-LC)
has the following sequence of SEQ ID NO 2:
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

As used herein, the term “Cetuximab antibody” encompasses Cetuximab and any antibody or antibody fragment that recognizes and specifically binds EGFR and has at least a heavy chain domain and light chain variable domain having at least 80% identity (e.g., at least 85%, 90%, 91%, 92%, 93%, 94%, 95%, 96%, 97%, 98%, 99%, or 100% identity) to a sequence of Cetuximab, preferably to SEQ ID NO: 1 or SEQ ID NO: 2. As used herein, the term “Cetuximab antibody” or Cetuximab also encompasses any antibody or antibody fragment that recognizes and specifically binds EGFR and has at least a heavy chain domain or light chain domain having at least 80% identity (e.g., at least 85%, 90%, 91%, 92%, 93%, 94%, 95%, 96%, 97%, 98%, 99%, or 100% identity) to a sequence of Cetuximab, preferably to SEQ ID NO: 1 or SEQ ID NO: 2, respectively.

In a preferred embodiment, a Cetuximab antibody is Cetuximab as defined in the prior art. Preferably, a Cetuximab antibody is Cetuximab as defined in (1) Li et al., 2005, op. cit.; (2) Dubois et al., 2008, op. cit.; (3) Cetuximab at HVIGT database, www.imgt.org/3Dstructure-DB/cgi/details.cgi?pdbcode=7906; (4) Ayoub et al., 2013, v op. cit.; or (5) Cetuximab at DrugBank (https://www.drugbank.ca/drugs/DB00002).

In a preferred embodiment, a Cetuximab antibody refers to an antibody including a Cetuximab heavy chain or Cetuximab light chain. In another preferred embodiment, a Cetuximab antibody refers to an antibody including a Cetuximab heavy chain and a Cetuximab light chain. In a preferred embodiment, a Cetuximab antibody includes a heavy chain having at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 1.

In a preferred embodiment, a Cetuximab antibody includes a light chain having at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 2

In a preferred embodiment, the Cetuximab antibody includes a heavy chain having at least 80% identity with SEQ ID NO: 1. In another embodiments, the Cetuximab antibody includes a light chain having at least 80% identity with SEQ ID NO: 2. In a preferred embodiment, the Cetuximab antibody includes a heavy chain having at least 80% identity with SEQ ID NO: 1, and the Cetuximab antibody includes a light chain having at least 80% identity with SEQ ID NO: 2.

In a preferred embodiment, the Cetuximab antibody comprises two heavy chains having at least 80% identity with SEQ ID NO: 1 and two light chains having at least 80% identity with SEQ ID NO: 2.

In a preferred embodiment, the Cetuximab antibody consists of a heavy chain having at least 80% identity with SEQ ID NO: 1. In another embodiments, the Cetuximab antibody consists of a light chain having at least 80% identity with SEQ ID NO: 2. In a preferred embodiment, the Cetuximab antibody consists of a heavy chain having at least 80% identity with SEQ ID NO: 1 and a light chain having at least 80% identity with SEQ ID NO: 2. In a preferred embodiment, the Cetuximab antibody consists of two heavy chains having at least 80% identity with SEQ ID NO: 1 and two light chains having at least 80% identity with SEQ ID NO: 2. In a preferred embodiment, the Cetuximab antibody includes a heavy chain having at least 90% identity with SEQ ID NO: 1. In another embodiments, the Cetuximab antibody includes a light chain having at least 90% identity with SEQ ID NO: 2. In a preferred embodiment, the Cetuximab antibody includes a heavy chain having at least 90% identity with SEQ ID NO: 1, and the Cetuximab antibody includes a light chain having at least 90% identity with SEQ ID NO: 2.

In a preferred embodiment, the Cetuximab antibody comprises two heavy chains having at least 90% identity with SEQ ID NO: 1 and two light chains having at least 90% identity with SEQ ID NO: 2.

In a preferred embodiment, the Cetuximab antibody consists of a heavy chain having at least 90% identity with SEQ ID NO: 1. In another embodiments, the Cetuximab antibody consists of a light chain having at least 90% identity with SEQ ID NO: 2. In a preferred embodiment, the Cetuximab antibody consists of a heavy chain having at least 90% identity with SEQ ID NO: 1 and a light chain having at least 90% identity with SEQ ID NO: 2. In a preferred embodiment, the Cetuximab antibody consists of two heavy chains having at least 90% identity with SEQ ID NO: 1 and two light chains having at least 90% identity with SEQ ID NO: 2.

In a preferred embodiment, the Cetuximab antibody includes a heavy chain having at least 95% identity with SEQ ID NO: 1. In another embodiments, the Cetuximab antibody includes a light chain having at least 95% identity with SEQ ID NO: 2. In a preferred embodiment, the Cetuximab antibody includes a heavy chain having at least 95% identity with SEQ ID NO: 1, and the Cetuximab antibody includes a light chain having at least 95% identity with SEQ ID NO: 2.

In a preferred embodiment, the Cetuximab antibody comprises two heavy chains having at least 95% identity with SEQ ID NO: 1 and two light chains having at least 95% identity with SEQ ID NO: 2.

In a preferred embodiment, the Cetuximab antibody consists of a heavy chain having at least 95% identity with SEQ ID NO: 1. In another embodiments, the Cetuximab antibody consists of a light chain having at least 95% identity with SEQ ID NO: 2. In a preferred embodiment, the Cetuximab antibody consists of a heavy chain having at least 95% identity with SEQ ID NO: 1 and a light chain having at least 95% identity with SEQ ID NO: 2. In a preferred embodiment, the Cetuximab antibody consists of two heavy chains having at least 95% identity with SEQ ID NO: 1 and two light chains having at least 95% identity with SEQ ID NO: 2. In a preferred embodiment, the Cetuximab antibody includes a heavy chain having at least 99% identity with SEQ ID NO: 1. In another embodiments, the Cetuximab antibody includes a light chain having at least 99% identity with SEQ ID NO: 2. In a preferred embodiment, the Cetuximab antibody includes a heavy chain having at least 99% identity with SEQ ID NO: 1, and the Cetuximab antibody includes a light chain having at least 99% identity with SEQ ID NO: 2.

In a preferred embodiment, the Cetuximab antibody comprises of two heavy chains having at least 99% identity with SEQ ID NO: 1 and two light chains having at least 99% identity with SEQ ID NO: 2.

In a preferred embodiment, the Cetuximab antibody consists of a heavy chain having at least 99% identity with SEQ ID NO: 1. In another embodiments, the Cetuximab antibody consists of a light chain having at least 99% identity with SEQ ID NO: 2. In a preferred embodiment, the Cetuximab antibody consists of a heavy chain having at least 99% identity with SEQ ID NO: 1 and a light chain having at least 99% identity with SEQ ID NO: 2. In a preferred embodiment, the Cetuximab antibody consists of two heavy chains having at least 99% identity with SEQ ID NO: 1 and two light chains having at least 99% identity with SEQ ID NO: 2.

In a preferred embodiment, the Cetuximab antibody includes a heavy chain having at least 100% identity with SEQ ID NO: 1. In another embodiments, the Cetuximab antibody includes a light chain having at least 100% identity with SEQ ID NO: 2. In a preferred embodiment, the Cetuximab antibody includes a heavy chain having at least 100% identity with SEQ ID NO: 1, and the Cetuximab antibody includes a light chain having at least 100% identity with SEQ ID NO: 2.

In a more preferred embodiment, the Cetuximab antibody comprises two heavy chains both of SEQ ID NO: 1, and two light chains both of SEQ ID NO: 2.

In a preferred embodiment, the Cetuximab antibody consists of a heavy chain having at least 100% identity with SEQ ID NO: 1. In another embodiments, the Cetuximab antibody consists of a light chain having at least 100% identity with SEQ ID NO: 2. In a preferred embodiment, the Cetuximab antibody consists of a heavy chain having at least 100% identity with SEQ ID NO: 1 and a light chain having at least 100% identity with SEQ ID NO: 2. In a more preferred embodiment, the Cetuximab antibody consists of two heavy chains both of SEQ ID NO: 1, and two light chains both of SEQ ID NO: 2. In a preferred embodiment, the Cetuximab antibody is a Cetuximab full antibody or a Cetuximab antibody fragment, such as a Fab, Fab″, F(ab′)₂, Fd, Fv or a single chain Fv (scFv). Cetuximab full antibody is more preferred, since it leads to a high level of Fc-mediated effects.

In a preferred embodiment, the Cetuximab antibody is a monospecific or multispecific antibody such as a bispecific antibody. More preferably, the Cetuximab antibody is a monospecific antibody.

In a preferred embodiment, the Cetuximab antibody comprises at least one light chain and at least one heavy chain. In a preferred embodiment, the Cetuximab antibody comprises two light chains and two heavy chains.

In a preferred embodiment, the Cetuximab antibody comprises (i) two heavy chains both of SEQ ID NO: 1, and two light chains both of SEQ ID NO: 2 or (ii) one heavy chain of SEQ ID NO: 1, and one light chain of SEQ ID NO: 2. In a more preferred embodiment, the Cetuximab antibody comprises two heavy chains both of SEQ ID NO: 1, and two light chains both of SEQ ID NO: 2.

In a preferred embodiment, the dsRBD of the recombinant protein of the invention is bound to the N terminus of the light chain of the Cetuximab antibody (CTX-LC-dsRBD) or the dsRBD is bound to the C terminus of the heavy chain of the Cetuximab antibody (CTX-HC-dsRBD). In a certain preferred embodiment, the dsRBD of the recombinant protein of the invention is bound to the N terminus of the light chain of the Cetuximab antibody (CTX-LC-dsRBD). In another preferred embodiment, the dsRBD is bound to the C terminus of the heavy chain of the Cetuximab antibody (CTX-HC-dsRBD).

The term “N terminus of the light chain” as used herein preferably refers to amino acids 1-24 at the N terminus of the light chain. Preferably, the N terminus of the light chain refers to the first N terminal amino acid of the light chain. More preferably, the N terminus of the light chain refers to the amine group of the first N terminal amino acid of the light chain. Most preferably, the N terminus of the light chain refers to the amine group linked to the alpha carbon atom of the first N terminal amino acid of the light chain.

The term “C terminus of the heavy chain” as used herein refers to amino acids ranging from amino acid position 416 to the last amino acid of the heavy chain. Preferably, the N terminus of the light chain refers to the last C terminal amino acid of the heavy chain. More preferably, the C terminus of the heavy chain refers to the carboxyl group of the last C terminal amino acid of the heavy chain. Again more preferably, the C terminus of the heavy chain refers to the carboxyl group linked to the alpha carbon atom of the last C terminal amino acid of the heavy chain.

With these attachment sites, dsRBD included in the recombinant protein of the invention did not interfere with protein A binding of the Cetuximab antibody and therefore allows an efficient purification process of the recombinant protein (see Examples).

Based on data with these specific attachment sites, it could be shown that dsRBD does also not interfere with binding of the recombinant protein to EGFR, and Fc-mediated immune responses in vivo will thus be induced.

By means of ELISA (cf. Examples), evidence could be provided that the recombinant protein of the invention comprising a dsRNA binding domain bound to the N terminus of the light chain of the Cetuximab antibody (CTX-LC-dsRBD) is capable of binding to its receptor EGFR and is thus biologically active. Conformation and folding of the Cetuximab antibody is not impaired by the fused dsRBD.

With the dsRBD of the recombinant protein of the invention bound to the N terminus of the light chain of the Cetuximab antibody (CTX-LC-dsRBD), the Fc region of the Cetuximab is not only available for purification with protein A, and for antibody-dependent cell-mediated cytotoxicity (ADCC), an important component of the anti-tumor immune reaction invoked by the treatment.

In a certain preferred embodiment, the dsRBD of the recombinant protein of the invention is bound to the N terminus of the light chain of the Cetuximab antibody (CTX-LC-dsRBD), and the Cetuximab antibody consists of a heavy chain having at least 100% identity with SEQ ID NO: 1 and a light chain having at least 100% identity with SEQ ID NO: 2.

In a certain preferred embodiment, the dsRBD of the recombinant protein of the invention is bound to the C terminus of the heavy chain of the Cetuximab antibody (CTX-LC-dsRBD), and the Cetuximab antibody consists of a heavy chain having at least 100% identity with SEQ ID NO: 1 and a light chain having at least 100% identity with SEQ ID NO: 2.

In a preferred embodiment, the dsRBD and the Cetuximab antibody are covalently bound.

In a further preferred embodiment, the dsRBD and the Cetuximab antibody are covalently bound via a spacer peptide. Preferably, said spacer peptide is a flexible spacer peptide. In a preferred embodiment, said spacer peptide is an oligopeptide of at least 15 amino acids, wherein said at least 15 amino acids are selected from the group consisting of Gly, Ser, Thr, Ala, Lys and Glu. In another preferred embodiment, said spacer peptide is an oligopeptide of at least 15 amino acids comprising an amino acid selected from the group consisting of Gly, Ser, Thr, Ala, Lys and Glu.

In another preferred embodiment, said dsRNA binding domain (dsRBD) and said the Cetuximab antibody are covalently bound via a Glycine-Serine spacer peptide. Preferably said Glycine-Serine spacer peptide comprises the peptide (Gly₄Ser)ₙ. In a preferred embodiment, said n is preferably 1, 2, 3 or 4. More preferably said n is 3, i.e. said spacer peptide comprises the peptide (Gly₄Ser)₃. Said spacer does neither interfere with a) binding of the Cetuximab antibody to EGFR nor with b) binding of the dsRBD to dsRNA or polyIC.

In another preferred embodiment, the dsRBD is bound to the light chain of the Cetuximab antibody (CTX-LC-dsRBD) via said spacer peptide, wherein said spacer peptide has an amino acid sequence of SEQ ID NO: 3 (GGGGSGGGGSGGGGS); or the dsRBD is bound to the heavy chain of the Cetuximab antibody (CTX-HC-dsRBD) via said spacer peptide, wherein said spacer peptide has an amino acid sequence of SEQ ID NO: 4 (GPGGGGSGGGGSGGGGS). In another preferred embodiment, the dsRBD is bound to the light chain of the Cetuximab antibody (CTX-LC-dsRBD) via said spacer peptide, wherein said spacer peptide has an amino acid sequence of SEQ ID NO: 3 (GGGGSGGGGSGGGGS). In another preferred embodiment, the dsRBD is bound to the heavy chain of the Cetuximab antibody (CTX-HC-dsRBD) via said spacer peptide, wherein said spacer peptide has an amino acid sequence of SEQ ID NO: 4 (GPGGGGSGGGGSGGGGS).

In another preferred embodiment, the dsRBD is bound to the N terminus of the light chain of the Cetuximab antibody (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of the Cetuximab antibody (CTX-HC-dsRBD) via said spacer peptide, wherein said spacer peptide has an amino acid sequence of SEQ ID NO: 4 (GPGGGGSGGGGSGGGGS). In a further preferred embodiment, the dsRBD is bound to the N terminus of the light chain of the Cetuximab antibody (CTX-LC-dsRBD) via said spacer peptide, wherein said spacer peptide has an amino acid sequence of SEQ ID NO: 3 (GGGGSGGGGSGGGGS). In another preferred embodiment, the dsRBD is bound to the C terminus of the heavy chain of the Cetuximab antibody (CTX-HC-dsRBD) via said spacer peptide, wherein said spacer peptide has an amino acid sequence of SEQ ID NO: 4 (GPGGGGSGGGGSGGGGS).

In another preferred embodiment, the dsRBD is bound to the N terminus of the light chain of the Cetuximab antibody (CTX-LC-dsRBD) via said spacer peptide, wherein said spacer peptide has an amino acid sequence of SEQ ID NO: 3 (GGGGSGGGGSGGGGS), and the Cetuximab antibody consists of a heavy chain having at least 100% identity with SEQ ID NO: 1 and a light chain having at least 100% identity with SEQ ID NO: 2.

In another preferred embodiment, the dsRBD is bound to the C terminus of the heavy chain of the Cetuximab antibody (CTX-HC-dsRBD) via said spacer peptide, wherein said spacer peptide has an amino acid sequence of SEQ ID NO: 4 (GPGGGGSGGGGSGGGGS), and the Cetuximab antibody consists of a heavy chain having at least 100% identity with SEQ ID NO: 1 and a light chain having at least 100% identity with SEQ ID NO: 2.

In another preferred embodiment, said dsRNA binding domain of the recombinant protein comprises one or more double-stranded RNA-binding motifs (dsRBm). Preferably, said dsRNA binding domain comprises two dsRBm. In another preferred embodiment, said dsRNA binding domain comprises two tandem linked dsRNA-binding motifs (dsRBm) both with an α-β-β-β-α fold.

Preferably, the dsRBD is a human dsRBD, i.e. it originates from a human protein. Thus, for example, immunogenicity is minimized.

In another preferred embodiment, said dsRBD is capable of binding dsRNA having a length from about 30 bp to about 80 bp. In another preferred embodiment, said dsRBD binds dsRNA in a sequence independent fashion.

In another preferred embodiment, said one or more dsRBm are selected from a dsRBm of dsRNA dependent protein kinase (PKR), TRBP, PACT, Staufen, NFAR1, NFARZ, SPNR, RHA and NREBP. In another preferred embodiment, at least one of said one or more dsRBm is an amino acid sequence of a dsRNA dependent protein kinase (PKR), preferably of human PKR (hPKR). In another preferred embodiment, at least one of said one or more dsRBm comprises an amino acid sequence of a dsRNA dependent protein kinase (PKR), preferably of human PKR (hPKR). In another preferred embodiment, at least one of said one or more dsRBm is a dsRBm of a dsRNA dependent protein kinase (PKR), preferably of human PKR (hPKR).

The term dsRNA dependent protein kinase (PKR) (also called dsRNA activated protein kinase, protein kinase R (PKR), interferon-induced dsRNA-activated protein kinase, or eukaryotic translation initiation factor 2-alpha kinase 2 (EIF2AK2)) as used herein refers to an enzyme that is encoded by the Eif2ak2 gene. The term human dsRNA dependent protein kinase (hPKR) as used herein refers to an enzyme that is encoded by the human Eif2ak2 gene including transcript variants encoding isoforms as mentioned in gene or protein data bases (e.g. NCBI Gene ID: 5610, UniProt P19525).

In another preferred embodiment, each of said one or more dsRBm comprises an amino acid sequence of a PKR, preferably of human PKR (hPKR). In another preferred embodiment, each of said one or more dsRBm is an amino acid sequence of a PKR, preferably of human PKR (hPKR). In another preferred embodiment, each of said one or more dsRBm is a dsRBm of a PKR, preferably of human PKR (hPKR).

In another preferred embodiment, said dsRBD comprises two dsRBm, wherein at least one dsRBm comprises an amino acid sequence of a PKR, preferably of hPKR. In another preferred embodiment, said dsRBD comprises two dsRBm, wherein at least one dsRBm is an amino acid sequence of a PKR, preferably of hPKR. In another preferred embodiment, said dsRBD comprises two dsRBm, wherein at least one dsRBm is a dsRBm of a PKR, preferably of hPKR.

In another preferred embodiment, said dsRBD comprises two dsRBm, wherein both dsRBm comprise an amino acid sequence of a PKR, preferably of hPKR. In another preferred embodiment, said dsRBD comprises two dsRBm, wherein both dsRBm are an amino acid sequence of a PKR, preferably of hPKR. In another preferred embodiment, said dsRBD comprises two dsRBm, wherein both dsRBm are dsRBm of a PKR, preferably of hPKR.

In another preferred embodiment, said dsRBD and said Cetuximab antibody are covalently bound via a spacer peptide, wherein said spacer peptide comprises the peptide (Gly₄Ser), wherein n is 1, 2, 3, or 4, preferably 3, and said dsRBD comprises two dsRBm, wherein both dsRBm are dsRBm of a PKR, preferably of hPKR. In another preferred embodiment, the dsRBD is bound to the N terminus of the light chain of the Cetuximab antibody (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of the Cetuximab antibody (CTX-HC-dsRBD) via said spacer peptide, wherein said spacer peptide has an amino acid sequence of SEQ ID NO: 4 (GPGGGGSGGGGSGGGGS), and said dsRBD comprises two dsRBm, wherein both dsRBm are dsRBm of a PKR, preferably of hPKR.

In another preferred embodiment, said dsRBD comprises amino acid residues 6-169 of PKR, preferably hPKR. In another preferred embodiment, said dsRBD consists of amino acid residues 6-169 of PKR, preferably hPKR. In another preferred embodiment, said dsRBD comprises full length PKR, preferably hPKR. In another preferred embodiment, said dsRBD consists of full length PKR, preferably hPKR.

In another preferred embodiment, said dsRBD comprises amino acid residues 6-169 of hPKR, wherein cysteine at position 121 and 135 is exchanged by a non-cysteine amino acid. In another preferred embodiment, said dsRBD consists of amino acid residues 6-169 of PKR, wherein cysteine at position 121 and 135 is exchanged by a non-cysteine amino acid. In another preferred embodiment, said dsRBD comprises full length hPKR, wherein cysteine at position 121 and 135 is exchanged by a non-cysteine amino acid. In another preferred embodiment, said dsRBD consists of full length hPKR, wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In certain embodiments, the dsRNA binding domain comprises amino acid residues 1-197 or 1-169 of human PKR. In certain embodiments, the dsRNA binding domain consists of amino acid residues 1-197 or 1-169 of human PKR. In certain embodiments, the dsRNA binding domain comprises amino acid residues 1-169 of human PKR. In certain embodiments, the dsRNA binding domain consists of amino acid residues 1-169 of human PKR.

In certain embodiments, the dsRNA binding domain comprises amino acid residues 1-197 or 1-169 of human PKR, wherein cysteine at position 121 and 135 is exchanged by a non-cysteine amino acid. In certain embodiments, the dsRNA binding domain consists of amino acid residues 1-197 or 1-169 of human PKR, wherein cysteine at position 121 and 135 is exchanged by a non-cysteine amino acid. In certain embodiments, the dsRNA binding domain comprises amino acid residues 1-169 of human PKR, wherein cysteine at position 121 and 135 is exchanged by a non-cysteine amino acid. In certain embodiments, the dsRNA binding domain consists of amino acid residues 1-169 of human PKR, wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In a very preferred certain embodiments, the dsRNA binding domain comprises amino acid residues 1-168 of human PKR, wherein cysteine at position 121 and 135 is exchanged by a non-cysteine amino acid. In certain embodiments, the dsRNA binding domain consists of amino acid residues 1-168 of human PKR, wherein cysteine at position 121 and 135 is exchanged by a non-cysteine amino acid. In a very preferred certain embodiments, the dsRNA binding domain (dsRBD) comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In certain embodiments, the dsRNA binding domain consists of amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid.

In a very preferred certain embodiments, the dsRNA binding domain (dsRBD) comprises amino acid residues 1-168 of human PKR or a homolog thereof, wherein in said homolog at least 80% of said amino acid residues are conserved, wherein F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In certain embodiments, the dsRNA binding domain consists of amino acid residues 1-168 of human PKR or a homolog thereof, wherein in said homolog at least 80% of said amino acid residues are conserved, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid.

In a very preferred certain embodiments, the dsRNA binding domain (dsRBD) comprises amino acid residues 1-168 of human PKR or a homolog thereof, wherein in said homolog amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In certain embodiments, the dsRNA binding domain consists of amino acid residues 1-168 of human PKR or a homolog thereof, wherein in said homolog amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid.

In a very preferred certain embodiments, the dsRNA binding domain (dsRBD) comprises amino acid residues 1-168 of human PKR or a homolog thereof, wherein in said homolog at least 80% of said amino acid residues are conserved, wherein in said homolog at least 80% of said amino acid residues are conserved, wherein F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In certain embodiments, the dsRNA binding domain consists of amino acid residues 1-168 of human PKR or a homolog thereof, wherein in said homolog at least 80% of said amino acid residues are conserved, wherein in said homolog at least 80% of said amino acid residues are conserved, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid.

Preferably at least 80%, more preferably at least 85%, again more preferably at least 90%, again more preferably at least 95%, again more preferably at least 99% of the amino acid residues in said homolog are conserved.

Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In another preferred embodiment, said dsRBD comprises two dsRBm, wherein one dsRBm (dsRBm1) consists of an amino acid sequence of residues 6-79 of hPKR or a homolog thereof; and the other dsRBm (dsRBm2) consists of an amino acid sequence of residues 96-169 of hPKR or a homolog thereof, wherein R39, F41, S59, K60, K61 and K64 are conserved in the homolog of dsRBm1, and F131, K150 and K154 are conserved in the homolog of dsRBm2. In another preferred embodiment, said dsRBD comprises two dsRBm, wherein one dsRBm (dsRBm1) consists of an amino acid sequence of residues 6-79 of hPKR or a homolog thereof; and the other dsRBm (dsRBm2) consists of an amino acid sequence of residues 96-168 of hPKR or a homolog thereof, wherein R39, F41, S59, K60, K61 and K64 are conserved in the homolog of dsRBm1, and F131, K150 and K154 are conserved in the homolog of dsRBm2, and wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In another preferred embodiment, said dsRBD comprises two dsRBm, wherein one dsRBm (dsRBm1) consists of an amino acid sequence of residues 6-79 of hPKR or a homolog thereof; and the other dsRBm (dsRBm2) consists of an amino acid sequence of residues 96-169 of hPKR or a homolog thereof, wherein F10, F43, V45, I47, A71, V72, R39, F41, S59, K60, K61 and K64 are conserved in the homolog of dsRBm1, and Y101, Y133, C135, M137, A161, F131, K150 and K154 are conserved in the homolog of dsRBm2.

In another preferred embodiment, said dsRBD comprises two dsRBm, wherein one dsRBm (dsRBm1) consists of an amino acid sequence of residues 6-79 of hPKR or a homolog thereof; and the other dsRBm (dsRBm2) consists of an amino acid sequence of residues 96-169 of hPKR or a homolog thereof, wherein F10, F43, V45, I47, A71, V72, R39, F41, S59, K60, K61 and K64 are conserved in the homolog of dsRBm1, and Y101, Y133, C135, M137, A161, F131, K150 and K154 are conserved in the homolog of dsRBm2, and wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said non-cysteine amino acid is alanine or valine.

In another preferred embodiment, said dsRBD comprises two dsRBm, wherein one dsRBm (dsRBm1) consists of an amino acid sequence of residues 6-79 of hPKR or a homolog thereof; and the other dsRBm (dsRBm2) consists of an amino acid sequence of residues 96-169 of hPKR or a homolog thereof, wherein amino acid residues 1-24, 39-50 and 58-69 are conserved in the homolog of dsRBm1, and Y101, Y133, C135, M137, A161, F131, K150 and K154 are conserved in the homolog of dsRBm2.

In another preferred embodiment, said dsRBD comprises two dsRBm, wherein one dsRBm (dsRBm1) consists of an amino acid sequence of residues 6-79 of hPKR or a homolog thereof; and the other dsRBm (dsRBm2) consists of an amino acid sequence of residues 96-169 of hPKR or a homolog thereof, wherein amino acid residues 1-24, 39-50 and 58-69 are conserved in the homolog of dsRBm1, and Y101, Y133, C135, M137, A161, F131, K150 and K154 are conserved in the homolog of dsRBm2, and wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said non-cysteine amino acid is alanine or valine.

In another preferred embodiment, said dsRBD comprises two dsRBm, wherein one dsRBm consists of an amino acid sequence of residues 6-79 of hPKR and the other dsRBm consists of an amino acid sequence of residues 96-169 of hPKR.

In another preferred embodiment, said dsRBD comprises two dsRBm, wherein one dsRBm consists of an amino acid sequence of residues 6-79 of hPKR and the other dsRBm consists of an amino acid sequence of residues 96-169 of hPKR, wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said non-cysteine amino acid is alanine or valine.

In another preferred embodiment, said dsRBD comprises two dsRBm, wherein one dsRBm (dsRBm1) consists of an amino acid sequence of SEQ ID NO: 5 or a homolog thereof; and the other dsRBm (dsRBm2) consists of an amino acid sequence SEQ ID NO: 6 or a homolog thereof, wherein R39, F41, S59, K60, K61 and K64 are conserved in the homolog of dsRBm1, and F131, K150 and K154 are conserved in the homolog of dsRBm2.

In another preferred embodiment, said dsRBD comprises two dsRBm, wherein one dsRBm (dsRBm1) consists of an amino acid sequence of SEQ ID NO: 5 or a homolog thereof; and the other dsRBm (dsRBm2) consists of an amino acid sequence of SEQ ID NO: 6 or a homolog thereof, wherein F10, F43, V45, I47, A71, V72, R39, F41, S59, K60, K61 and K64 are conserved in the homolog of dsRBm1, and Y101, Y133, C135, M137, A161, F131, K150 and K154 are conserved in the homolog of dsRBm2.

In another preferred embodiment, said dsRBD comprises two dsRBm, wherein one dsRBm (dsRBm1) consists of an amino acid sequence SEQ ID NO: 5 or a homolog thereof; and the other dsRBm (dsRBm2) consists of an amino acid sequence of SEQ ID NO: 6 or a homolog thereof, wherein amino acid residues 1-24, 39-50 and 58-69 are conserved in the homolog of dsRBm1, and Y101, Y133, C135, M137, A161, F131, K150 and K154 are conserved in the homolog of dsRBm2.

In another preferred embodiment, said dsRBD comprises two dsRBm, wherein one dsRBm consists of an amino acid sequence of SEQ ID NO: 5 and the other dsRBm consists of an amino acid sequence of SEQ ID NO: 6.

SEQ ID NO: 5 has the sequence of LSAGF-FMEELNTYRQ-KQGVVLKYQE-LPNSGPPHDR-RFTFQVIIDG-REFPEGEGRS-KKEAKNAAAK-LAVEILNKE

SEQ ID NO: 6 has the sequence of LSMG-NYIGLINRIA-QKKRLTVNYE-QVASGVHGPE-GFHYKVKMGQ-KEYSIGTGST-KQEAKQLAAK-LAYLQILSE

In another preferred embodiment, said dsRBD comprises two dsRBm, wherein said dsRBm are linked via a linker consisting of 15-25, preferably 16-20 amino acids. In another preferred embodiment, said linker consists of residues 80-95 of PKR, preferably hPKR. In another preferred embodiment, said linker consists of an amino acid sequence of SEQ ID NO: 7. SEQ ID NO: 7 has the sequence of KKAVSPLLLTTTNSSEG.

In another preferred embodiment, said dsRBD has the sequence of SEQ ID NO: 8. SEQ ID NO: 8 has the sequence of MMAGDLSAGFFMEELNTYRQKQGVV LKYQELPNSGPPHDRRFTFQVIIDGREFPEGEGRSKKEAKNAAAKLAVEILNKEKKAV SPLLLTTTNSSEGLSMGNYIGLINRIAQKKRLTVNYEQVASGVHGPEGFHYKVKMGQ KEYSIGTGSTKQEAKQLAAKLAYLQILSE

In another preferred embodiment, said dsRBD and said Cetuximab are covalently bound via a spacer peptide, wherein said spacer peptide comprises the peptide (Gly₄Ser)₃ and said dsRNA binding domain comprises amino acid residues 1-168 of human PKR, wherein cysteine at position 121 and 135 is exchanged by a non-cysteine amino acid. In another preferred embodiment, said dsRBD is bound to the N terminus of the light chain of Cetuximab (CTX-LC-dsRBD) via a spacer peptide, wherein said spacer peptide comprises the peptide (Gly₄Ser)₃ and said dsRNA binding domain comprises amino acid residues 1-168 of human PKR, wherein cysteine at position 121 and 135 is exchanged by a non-cysteine amino acid. In certain embodiments, the dsRNA binding domain consists of amino acid residues 1-168 of human PKR, wherein cysteine at position 121 and 135 is exchanged by a non-cysteine amino acid. In another preferred embodiment, the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRNA binding domain comprises amino acid residues 1-168 of human PKR, wherein cysteine at position 121 and 135 is exchanged by a non-cysteine amino acid.

In another preferred embodiment, said dsRBD and said Cetuximab are covalently bound via a spacer peptide, wherein said spacer peptide comprises the peptide (Gly₄Ser)₃ and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In another preferred embodiment, said dsRBD is bound to the N terminus of the light chain of Cetuximab (CTX-LC-dsRBD) via a spacer peptide, wherein said spacer peptide comprises the peptide (Gly₄Ser)₃ and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In certain embodiments, the dsRNA binding domain consists of amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In another preferred embodiment, the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In another preferred embodiment, said dsRBD and said Cetuximab are covalently bound via a spacer peptide, wherein said spacer peptide comprises the peptide (Gly₄Ser)₃ and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In another preferred embodiment, said dsRBD is bound to the N terminus of the light chain of Cetuximab (CTX-LC-dsRBD) via a spacer peptide, wherein said spacer peptide comprises the peptide (Gly₄Ser)₃ and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In certain embodiments, the dsRNA binding domain consists of amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In another preferred embodiment, the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In another preferred embodiment, said dsRBD and said Cetuximab are covalently bound via a spacer peptide, wherein said spacer peptide comprises the peptide (Gly₄Ser)₃ and said dsRBD has the sequence of SEQ ID NO: 8.

In another preferred embodiment, the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRBD has the sequence of SEQ ID NO: 8.

In another preferred embodiment, the recombinant protein of the invention further comprises a cytolytic peptide. Preferably, said cytolytic peptide is Melittin or Candidalysin. In another preferred embodiment, said cytolytic peptide is positioned within the spacer peptide or at the N terminus of the recombinant protein.

In another preferred embodiment, the recombinant protein may further comprise a purification tag, such as His6 tag, for purification purposes. Preferably, the recombinant protein is free of contaminating dsRNA remaining from the manufacturing process of the recombinant protein.

In another preferred embodiment, the dsRBD is bound to the N terminus of the light chain of Cetuximab (CTX-LC-dsRBD) having SEQ ID NO: 1 via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) having SEQ ID NO: 2 via said spacer peptide, wherein said spacer peptide has an amino acid sequence of SEQ ID NO: 4 (GPGGGGSGGGGSGGGGS), said dsRBD has the sequence of SEQ ID NO: 8.

In another preferred embodiment, the dsRBD is bound to the N terminus of the light chain of Cetuximab (CTX-LC-dsRBD) having SEQ ID NO: 1 via said spacer peptide, wherein said spacer peptide has an amino acid sequence of SEQ ID NO: 3 (GGGGSGGGGSGGGGS). In another preferred embodiment, the dsRBD is bound to the C terminus of the heavy chain of Cetuximab (CTX-HC-dsRBD) having SEQ ID NO: 2 via said spacer peptide, wherein said spacer peptide has an amino acid sequence of SEQ ID NO: 4 (GPGGGGSGGGGSGGGGS), said dsRBD has the sequence of SEQ ID NO: 8.

In a preferred embodiment of the invention, the Cetuximab antibody comprises a heavy and a light chain of Cetuximab and dsRNA binding domain (dsRBD), said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR, wherein cysteine at position 121 and 135 is exchanged by a non-cysteine amino acid. In a preferred embodiment of the invention, the Cetuximab antibody comprises a heavy and a light chain of Cetuximab and dsRNA binding domain (dsRBD), said dsRNA binding domain is bound to the C terminus of said Cetuximab heavy chain of (CTX-HC-dsRBD) via a spacer peptide, wherein said spacer peptide comprises the peptide (Gly₄Ser)₃, and said dsRNA binding domain comprises amino acid residues 1-168 of human PKR, wherein cysteine at position 121 and 135 is exchanged by a non-cysteine amino acid. In a further preferred embodiment, said dsRNA binding domain consists of amino acid residues 1-168 of human PKR, wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In a preferred embodiment of the invention, the Cetuximab antibody comprises a heavy and a light chain of Cetuximab and dsRNA binding domain (dsRBD), said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In a preferred embodiment of the invention, the Cetuximab antibody comprises a heavy and a light chain of Cetuximab and dsRNA binding domain (dsRBD), said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In a preferred embodiment of the invention, the Cetuximab antibody comprises a heavy and a light chain of Cetuximab and dsRNA binding domain (dsRBD), said dsRNA binding domain is bound to the C terminus of said Cetuximab heavy chain of (CTX-HC-dsRBD) via a spacer peptide, wherein said spacer peptide comprises the peptide (Gly₄Ser)₃, and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a further preferred embodiment, said dsRNA binding domain consists of amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a preferred embodiment of the invention, the Cetuximab antibody comprises a heavy and a light chain of Cetuximab and dsRNA binding domain (dsRBD), said dsRNA binding domain is bound to the C terminus of said Cetuximab heavy chain of (CTX-HC-dsRBD) via a spacer peptide, wherein said spacer peptide comprises the peptide (Gly₄Ser)₃, and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In a further preferred embodiment, said dsRNA binding domain consists of amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In another preferred embodiment of the invention, the Cetuximab antibody comprises a heavy and a light chain of Cetuximab and a dsRBD, wherein the dsRBD is of SEQ ID NO: 8 and is bound to the N terminus of said Cetuximab light chain of (CTX-LC-dsRBD) via a spacer peptide, wherein said spacer peptide has an amino acid sequence of SEQ ID NO: 3 (GGGGSGGGGSGGGGS). In another preferred embodiment of the invention, the Cetuximab antibody comprises a Cetuximab heavy chain of SEQ ID NO: 1 and a Cetuximab light chain of SEQ ID NO: 2 and a dsRBD, wherein the dsRBD is of SEQ ID NO: 8 and is bound to the N terminus of said Cetuximab light chain of (CTX-LC-dsRBD) via a spacer peptide, wherein said spacer peptide has an amino acid sequence of SEQ ID NO: 3 (GGGGSGGGGSGGGGS).

In another preferred embodiment of the invention, the Cetuximab antibody comprises a heavy and a light chain of Cetuximab and a dsRBD, wherein the dsRBD is of SEQ ID NO: 8 and is bound to the C terminus of said Cetuximab heavy chain of (CTX-HC-dsRBD) via a spacer peptide, wherein said spacer peptide has an amino acid sequence of SEQ ID NO: 4. In another preferred embodiment of the invention, the Cetuximab antibody comprises a Cetuximab heavy chain of SEQ ID NO: 1 and a Cetuximab light chain of SEQ ID NO: 2 and a dsRBD, wherein the dsRBD is of SEQ ID NO: 8 and is bound to the C terminus of said Cetuximab heavy chain of (CTX-HC-dsRBD) via a spacer peptide, wherein said spacer peptide has an amino acid sequence of SEQ ID NO: 4.

In another preferred embodiment, the dsRBD is bound to the N terminus of the light chain of Cetuximab (CTX-LC-dsRBD) having SEQ ID NO: 1 via the spacer peptide, wherein said spacer peptide has an amino acid sequence of SEQ ID NO: 3 (GGGGSGGGGSGGGGS), wherein an apoplast signal peptide is bound to the N terminus of the dsRBD; or the dsRBD is bound to the C terminus of the heavy chain of Cetuximab (CTX-HC-dsRBD) having SEQ ID NO: 2 via the spacer peptide, wherein said spacer peptide has an amino acid sequence of SEQ ID NO: 4 (GPGGGGSGGGGSGGGGS), said dsRBD has the sequence of SEQ ID NO: 8, wherein an apoplast signal peptide is bound to the N terminus of the heavy chain of Cetuximab.

In another preferred embodiment, the dsRBD is bound to the N terminus of the light chain of Cetuximab (CTX-LC-dsRBD) having SEQ ID NO: 1 via said spacer peptide, wherein said spacer peptide has an amino acid sequence of SEQ ID NO: 3 (GGGGSGGGGSGGGGS), wherein an apoplast signal peptide is bound to the N terminus of the dsRBD. In another preferred embodiment, the dsRBD is bound to the C terminus of the heavy chain of Cetuximab (CTX-HC-dsRBD) having SEQ ID NO: 2 via said spacer peptide, wherein said spacer peptide has an amino acid sequence of SEQ ID NO: 4 (GPGGGGSGGGGSGGGGS), said dsRBD has the sequence of SEQ ID NO: 8, wherein an apoplast signal peptide is bound to the N terminus of the heavy chain of Cetuximab.

In a preferred embodiment of the invention, the Cetuximab antibody comprises a heavy and a light chain of Cetuximab and dsRNA binding domain (dsRBD), said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR, wherein cysteine at position 121 and 135 is exchanged by a non-cysteine amino acid, and a first apoplast signal peptide is bound to the N terminus of the dsRBD and a second apoplast peptide is bound to the N terminus of the Cetuximab heavy chain. In a preferred embodiment of the invention, the Cetuximab antibody comprises a heavy and a light chain of Cetuximab and dsRNA binding domain (dsRBD), said dsRNA binding domain is bound to the C terminus of said Cetuximab heavy chain of (CTX-HC-dsRBD) via a spacer peptide, wherein said spacer peptide comprises the peptide (Gly₄Ser)₃, and said dsRNA binding domain comprises amino acid residues 1-168 of human PKR, wherein cysteine at position 121 and 135 is exchanged by a non-cysteine amino acid, and a first apoplast signal peptide is bound to the N terminus of the dsRBD and a second apoplast peptide is bound to the N terminus of the Cetuximab heavy chain. Preferably, said first and second apoplast peptide are different from each other. More preferably, said first and second apoplast peptide are different endoglucanases. Again more preferably said first and second apoplast peptide are different endoglucanases from different species. Again more preferably said first and second apoplast peptide are different endoglucanases, wherein the first is from cotton and the second is from tobacco. Again more preferably said first and second apoplast peptide are different endoglucanases, wherein the first is of SEQ ID NO: 16 and the second is of SEQ ID NO: 17. Again more preferably said first and second apoplast peptide are different endoglucanases, wherein the first is of SEQ ID NO: 16 attached to the heavy chain and the second is of SEQ ID NO: 17 attached to the light chain.

In a preferred embodiment of the invention, the Cetuximab antibody comprises a heavy and a light chain of Cetuximab, dsRNA binding domain (dsRBD), and a first and a second apoplast signal peptide; said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid; said first apoplast signal peptide is bound to the N terminus of the dsRBD and a second apoplast peptide is bound to the N terminus of the Cetuximab heavy chain. In a preferred embodiment of the invention, the Cetuximab antibody comprises a heavy and a light chain of Cetuximab, a dsRNA binding domain (dsRBD), and a first apoplast signal peptide; said dsRNA binding domain is bound to the C terminus of said Cetuximab heavy chain of (CTX-HC-dsRBD) via a spacer peptide, wherein said spacer peptide comprises the peptide (Gly₄Ser)₃, and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid; said first apoplast signal peptide is bound to the N terminus of the dsRBD and said second apoplast peptide is bound to the N terminus of the Cetuximab heavy chain. Preferably, said first and second apoplast peptide are different from each other. More preferably, said first and second apoplast peptide are different endoglucanases. Again more preferably said first and second apoplast peptide are different endoglucanases from different species. Again more preferably said first and second apoplast peptide are different endoglucanases, wherein the first is from cotton and the second is from tobacco. Again more preferably said first and second apoplast peptide are different endoglucanases, wherein the first is of SEQ ID NO: 16 and the second is of SEQ ID NO: 17. Again more preferably said first and second apoplast peptide are different endoglucanases, wherein the first is of SEQ ID NO: 16 attached to the heavy chain and the second is of SEQ ID NO: 17 attached to the light chain. In another preferred embodiment of the invention, the Cetuximab antibody comprises a heavy and a light chain of Cetuximab and a dsRBD, wherein the dsRBD is of SEQ ID NO: 8 and is bound to the N terminus of said Cetuximab light chain of (CTX-LC-dsRBD) via a spacer peptide, wherein said spacer peptide has an amino acid sequence of SEQ ID NO: 3 (GGGGSGGGGSGGGGS), and a first apoplast signal peptide is bound to the N terminus of the dsRBD and a second apoplast peptide is bound to the N terminus of the Cetuximab heavy chain. In another preferred embodiment of the invention, the Cetuximab antibody comprises a Cetuximab heavy chain of SEQ ID NO: 1 and a Cetuximab light chain of SEQ ID NO: 2 and a dsRBD, wherein the dsRBD is of SEQ ID NO: 8 and is bound to the N terminus of said Cetuximab light chain of (CTX-LC-dsRBD) via a spacer peptide, wherein said spacer peptide has an amino acid sequence of SEQ ID NO: 3 (GGGGSGGGGSGGGGS), and a first apoplast signal peptide is bound to the N terminus of the dsRBD and a second apoplast peptide is bound to the N terminus of the Cetuximab heavy chain. In another preferred embodiment of the invention, the Cetuximab antibody comprises a Cetuximab heavy chain of SEQ ID NO: 1 and a Cetuximab light chain of SEQ ID NO: 2 and a dsRBD, wherein the dsRBD is of SEQ ID NO: 8 and is bound to the C terminus of said Cetuximab heavy chain of (CTX-HC-dsRBD) via a spacer peptide, wherein said spacer peptide has an amino acid sequence of SEQ ID NO: 4, and a first apoplast signal peptide is bound to the N terminus of the heavy chain and a second apoplast signal peptide is bound to the N terminus of the light chain. Preferably, said first and second apoplast peptide are different from each other.

In another preferred embodiment of the invention, the Cetuximab antibody comprises a heavy and a light chain of Cetuximab and a dsRBD, wherein the dsRBD is of SEQ ID NO: 8 and is bound to the C terminus of said Cetuximab heavy chain of (CTX-HC-dsRBD) via a spacer peptide, wherein said spacer peptide has an amino acid sequence of SEQ ID NO: 4, and a first apoplast signal peptide is bound to the N terminus of the heavy chain and a second apoplast signal peptide is bound to the N terminus of the light chain. In another preferred embodiment of the invention, the Cetuximab antibody comprises a Cetuximab heavy chain of SEQ ID NO: 1 and a Cetuximab light chain of SEQ ID NO: 2 and a dsRBD, wherein the dsRBD is of SEQ ID NO: 8 and is bound to the C terminus of said Cetuximab heavy chain of (CTX-HC-dsRBD) via a spacer peptide, wherein said spacer peptide has an amino acid sequence of SEQ ID NO: 4 and a first apoplast signal peptide is bound to the N terminus of the heavy chain and a second apoplast signal peptide is bound to the N terminus of the light chain. Preferably, said first and second apoplast peptide are different from each other. More preferably, said first and second apoplast peptide are different endoglucanases. Again more preferably said first and second apoplast peptide are different endoglucanases from different species. Again more preferably said first and second apoplast peptide are different endoglucanases, wherein the first is from cotton and the second is from tobacco. Again more preferably said first and second apoplast peptide are different endoglucanases, wherein the first is of SEQ ID NO: 16 and the second is of SEQ ID NO: 17. Again more preferably said first and second apoplast peptide are different endoglucanases, wherein the first is of SEQ ID NO: 16 attached to the heavy chain and the second is of SEQ ID NO: 17 attached to the light chain.

Preferably, said dsRNA binding domain consists of amino acid residues 1-168 of human PKR, wherein cysteine at position 121 and 135 is exchanged by a non-cysteine amino acid. Preferably, said dsRNA binding domain consists of amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

Preferably, said first and second apoplast peptide are different from each other. More preferably, one of said first and second apoplast peptide is of tobacco and the other is a cotton apoplast peptide. Again more preferably, one of said first and second apoplast peptide is of SEQ ID NO: 16 and the other is of SEQ ID NO. 17.

In a further aspect, the invention relates to a complex comprising the recombinant protein of the invention and dsRNA.

In a preferred embodiment, the dsRNA of the complex of the invention comprises polyinosinic-polycytidylic acid (polyIC). In another preferred embodiment, said dsRNA is selected from the group consisting of polyinosinic-polycytidylic acid (polyIC), polyinosinic-polycytidylic acid with poly-L-lysine and carboxymethylcellulose (poly(I,C)-LC), and polyinosinic-polycytidylic acid with poly-L-lysine (poly(I,C)-L). In another preferred embodiment, said dsRNA is polyinosinic-polycytidylic acid (polyIC).

In another preferred embodiment, said poly IC comprises at least 22 ribonucleotides in each strand. Preferably, said poly IC comprises 85-300 ribonucleotides in each strand.

In another preferred embodiment, said dsRNA is selected from polyIC, microRNA (miRNA), small interfering RNA (siRNA), and small hairpin RNA (shRNA). In another preferred embodiment, said dsRNA is from microRNA (miRNA). In another preferred embodiment, said dsRNA is small interfering RNA (siRNA). In another preferred embodiment, said dsRNA is small hairpin RNA (shRNA).

In another preferred embodiment, said dsRNA of said complex of the invention comprises at least one miRNA, siRNA or shRNA directed against a pro-oncogenic nucleic acid. In a certain embodiments, the dsRNA of the complex comprises at least one siRNA sequence directed against a pro-oncogenic mRNA, such as, but not limited to, Bcl-x1, Bcl-2, Mcl-1, Stat3, Pkb/Akt.

In another preferred embodiment, dsRNA of the complex of the invention comprises polyinosinic-polycytidylic acid (polyIC); said dsRBD and said Cetuximab are covalently bound via a spacer peptide, wherein said spacer peptide comprises the peptide (Gly₄Ser)₃ and said dsRNA binding domain comprises amino acid residues 1-168 of human PKR, wherein cysteine at position 121 and 135 is exchanged by a non-cysteine amino acid. In another preferred embodiment, said dsRBD is bound to the N terminus of the light chain of Cetuximab (CTX-LC-dsRBD) via a spacer peptide, wherein said spacer peptide comprises the peptide (Gly₄Ser)₃ and said dsRNA binding domain comprises amino acid residues 1-168 of human PKR, wherein cysteine at position 121 and 135 is exchanged by a non-cysteine amino acid. In another preferred embodiment, dsRNA of the complex of the invention comprises polyinosinic-polycytidylic acid (polyIC) and the dsRNA binding domain consists of amino acid residues 1-168 of human PKR, wherein cysteine at position 121 and 135 is exchanged by a non-cysteine amino acid. In another preferred embodiment, dsRNA of the complex of the invention comprises polyinosinic-polycytidylic acid (polyIC) and the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRNA binding domain comprises amino acid residues 1-168 of human PKR, wherein cysteine at position 121 and 135 is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In another preferred embodiment, dsRNA of the complex of the invention comprises polyinosinic-polycytidylic acid (polyIC); said dsRBD and said Cetuximab are covalently bound via a spacer peptide, wherein said spacer peptide comprises the peptide (Gly₄Ser)₃ and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In another preferred embodiment, said dsRBD is bound to the N terminus of the light chain of Cetuximab (CTX-LC-dsRBD) via a spacer peptide, wherein said spacer peptide comprises the peptide (Gly₄Ser)₃ and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In another preferred embodiment, dsRNA of the complex of the invention comprises polyinosinic-polycytidylic acid (polyIC) and the dsRNA binding domain consists of amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In another preferred embodiment, dsRNA of the complex of the invention comprises polyinosinic-polycytidylic acid (polyIC) and the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRNA binding domain comprises amino acid residues 1-168 of human PKR or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In another preferred embodiment, dsRNA of the complex of the invention comprises polyinosinic-polycytidylic acid (polyIC); said dsRBD and said Cetuximab are covalently bound via a spacer peptide, wherein said spacer peptide comprises the peptide (Gly₄Ser)₃ and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In another preferred embodiment, said dsRBD is bound to the N terminus of the light chain of Cetuximab (CTX-LC-dsRBD) via a spacer peptide, wherein said spacer peptide comprises the peptide (Gly₄Ser)₃ and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In another preferred embodiment, dsRNA of the complex of the invention comprises polyinosinic-polycytidylic acid (polyIC) and the dsRNA binding domain consists of amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In another preferred embodiment, dsRNA of the complex of the invention comprises polyinosinic-polycytidylic acid (polyIC) and the dsRBD is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), and said dsRNA binding domain comprises amino acid residues 1-168 of human PKR or a homolog thereof, wherein in said homolog 1-24, 39-50 and 58-69, Y101, Y133, C135, M137, A161, F131, K150 and K154 are conserved, and wherein cysteine at position 121 and 135 of said dsRBD is exchanged by a non-cysteine amino acid.

Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine. Preferably at least 80%, more preferably at least 85%, again more preferably at least 90%, again more preferably at least 95%, again more preferably at least 99% of the amino acid residues in said homolog are conserved.

In a preferred embodiment of the invention, dsRNA of the complex of the invention comprises, preferably is polyinosinic-polycytidylic acid (polyIC); the Cetuximab antibody comprises a heavy and a light chain of Cetuximab and dsRNA binding domain (dsRBD), said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR, wherein cysteine at position 121 and 135 is exchanged by a non-cysteine amino acid. In a further preferred embodiment, the dsRNA binding domain consists of amino acid residues 1-168 of human PKR, wherein cysteine at position 121 and 135 is exchanged by a non-cysteine amino acid. Preferably said non-cysteine amino acid is an alanine derivative.

Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In a preferred embodiment of the invention, dsRNA of the complex of the invention comprises, preferably is polyinosinic-polycytidylic acid (polyIC); the Cetuximab antibody comprises a heavy and a light chain of Cetuximab and dsRNA binding domain (dsRBD), said dsRNA binding domain is bound to the N terminus of said Cetuximab light chain of (CTX-LC-dsRBD) via a spacer peptide, wherein said spacer peptide comprises the peptide (Gly₄Ser)₃, and said dsRNA binding domain comprises amino acid residues 1-168 of human PKR, or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably, said dsRNA binding domain consists of amino acid residues 1-168 of human PKR, or a homolog thereof, wherein in said homolog F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine. In a preferred embodiment of the invention, dsRNA of the complex of the invention comprises, preferably is polyinosinic-polycytidylic acid (polyIC); the Cetuximab antibody comprises a heavy and a light chain of Cetuximab and a dsRBD, wherein the dsRBD is of SEQ ID NO: 8 and is bound to the N terminus of said Cetuximab light chain of (CTX-LC-dsRBD) via a spacer peptide, wherein said spacer peptide has an amino acid sequence of SEQ ID NO: 3 (GGGGSGGGGSGGGGS). In a preferred embodiment of the invention, dsRNA of the complex of the invention comprises, preferably is polyinosinic-polycytidylic acid (polyIC); the Cetuximab antibody comprises a Cetuximab heavy chain of SEQ ID NO: 1 and a Cetuximab light chain of SEQ ID NO: 2 and a dsRBD, wherein the dsRBD is of SEQ ID NO: 8 and is bound to the N terminus of said Cetuximab light chain of (CTX-LC-dsRBD) via a spacer peptide, wherein said spacer peptide has an amino acid sequence of SEQ ID NO: 3 (GGGGSGGGGSGGGGS).

In another preferred embodiment, said dsRNA in the complex of the invention is non-covalently associated with said dsRNA binding domain. In another preferred embodiment, said polyIC of the complex of the invention is non-covalently associated with said dsRNA binding domain. In another preferred embodiment, said miRNA, siRNA or shRNA of the complex of the invention is non-covalently associated with said dsRNA binding domain.

In another preferred embodiment, said dsRBD and said Cetuximab of the complex of the invention are covalently bound via a spacer peptide, wherein said spacer peptide comprises the peptide (Gly₄Ser)₃, said dsRBD has the sequence of SEQ ID NO: 8, and said dsRNA is polyIC.

In another preferred embodiment, the dsRBD in the complex of the invention is bound to the N terminus of the light chain of Cetuximab (CTX-LC-dsRBD) via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) via said spacer peptide, wherein said spacer peptide has an amino acid sequence of SEQ ID NO: 4 (GPGGGGSGGGGSGGGGS), said dsRBD has the sequence of SEQ ID NO: 8, and said dsRNA is polyIC.

In another preferred embodiment, the dsRBD in the complex of the invention is bound to the N terminus of the light chain of Cetuximab (CTX-LC-dsRBD) having SEQ ID NO: 1 via said spacer peptide, wherein said spacer peptide has an amino acid sequence of SEQ ID NO: 3 (GGGGSGGGGSGGGGS); or the dsRBD is bound to the C terminus of the heavy chain of Cetuximab (CTX-HC-dsRBD) having SEQ ID NO: 2 via said spacer peptide, wherein said spacer peptide has an amino acid sequence of SEQ ID NO: 4 (GPGGGGSGGGGSGGGGS), said dsRBD has the sequence of SEQ ID NO: 8, and said dsRNA is polyIC.

In a further aspect, the invention relates to a pharmaceutical composition comprising the recombinant protein or the complex of the invention and a pharmaceutically acceptable carrier. The pharmaceutical composition can be formulated for administration by any known method. The pharmaceutical composition of the invention may be formulated for intravenous, intra-brain (intracerebral), oral, intradermal, intramuscular, subcutaneous, transdermal, transmucosal, intranasal or intraocular administration.

In a further aspect, the present invention relates to the recombinant protein, complex or pharmaceutical composition of the invention for use in the treatment of cancer, wherein said cancer is characterized by EGFR-overexpressing cells. In another aspect, the invention relates to a method for treatment of cancer characterized by EGFR-overexpressing cells, said method comprises systemically administering to a patient the recombinant protein, complex or pharmaceutical composition of the invention.

In certain embodiments, the cancer characterized by EGFR-overexpressing cells is selected from non-small-cell-lung-carcinoma, breast cancer, glioblastoma, head and neck squamous cell carcinoma, gastric cancer, oesophageal cancer, colorectal cancer, adenocarcinoma, ovary cancer, cervical cancer, endometrial cancer, bladder cancer or prostate cancer, and metastases thereof.

In certain embodiments, the treatment further comprises administering immune cells, such as tumor-infiltrating T-cells (T-TILs), tumor specific engineered T-cells, or peripheral blood mononuclear cells (PBMCs).

In a further aspect, the invention relates to a vector comprising a nucleic acid sequence encoding the recombinant protein of the invention.

In a preferred embodiment, said vector includes a nucleic acid sequence encoding a light chain of a Cetuximab antibody as defined herein and a heavy chain of a Cetuximab antibody as defined herein and a nucleic acid sequence encoding a dsRBD as defined herein.

In a preferred embodiment, said vector includes SEQ ID NOs: 9, 10 and 11. In a preferred embodiment, said vector includes SEQ ID NOs: 10, 11 and 22.

In a preferred embodiment, said vector includes SEQ ID NOs: 10, 22 and one of 14 or 15. In a preferred embodiment, said vector includes SEQ ID NOs: 10, 22, 12, 13 and one of 14 or 15. In a preferred embodiment, said vector includes SEQ ID NOs: 10, 22, 12, 13 and one of 14 or 15, and at least one of 18, 19 or 20. In a preferred embodiment, said vector includes SEQ ID NOs: 10, 21, 12, 13, 18, 19 and 20. In a preferred embodiment, said vector includes SEQ ID NOs: 10, 21, 12, 13, 18, 19 and 20 and one of 14 or 15.

In a preferred embodiment, said vector includes SEQ ID NOs: 11, 22, 12, 13 and one of 14 or 15. In a preferred embodiment, said vector includes SEQ ID NOs: 11, 22, 12, 13 and one of 14 or 15, and at least one of 18, 19 or 20. In a preferred embodiment, said vector includes SEQ ID NOs: 11, 22, 12, 13, 18, 19 and 20. In a preferred embodiment, said vector includes SEQ ID NOs: 11, 22, 12, 13, 18, 19 and 20 and one of 14 or 15.

In a preferred embodiment, said vector includes SEQ ID NOs: 10, 11, 22, 12, 13, 14 and 15. In a preferred embodiment, said vector includes SEQ ID NOs: 10, 11, 22, 12, 13, 14 and 15, and at least one of 18, 19 or 20. In a preferred embodiment, said vector includes SEQ ID NOs: 10, 11, 22, 12, 13, 18, 19 and 20. In a preferred embodiment, said vector includes SEQ ID NOs: 10, 22, 21, 12, 13, 18, 19, 20, 14 and 15.

In a preferred embodiment, said vector includes SEQ ID NOs: 23 and 24. In a preferred embodiment, said vector includes SEQ ID NOs: 24 and 26. In a preferred embodiment, said vector includes SEQ ID NOs: 25 and 26 and either 23 or 24.

In a preferred embodiment, said vector includes SEQ ID NOs: 23 and 10. In a preferred embodiment, said vector includes SEQ ID NOs: 24 and 11. In a preferred embodiment, said vector includes SEQ ID NOs: 10 and 11 and either 23 or 24.

In a preferred embodiment, said vector includes SEQ ID NOs: 22, 23 and 25. In a preferred embodiment, said vector includes SEQ ID NOs: 22, 24 and 26. In a preferred embodiment, said vector includes SEQ ID NOs: 22, 25 and 26 and either 23 or 24.

In a preferred embodiment, said vector includes SEQ ID NOs: 22, 23 and 10. In a preferred embodiment, said vector includes SEQ ID NOs: 22, 24 and 11. In a preferred embodiment, said vector includes SEQ ID NOs: 22, 10 and 11 and either 23 or 24. In a preferred embodiment, said vector includes SEQ ID NO: 27. In a preferred embodiment, said vector includes SEQ ID NO: 28. In a preferred embodiment, said vector includes SEQ ID NOs: 27. In a preferred embodiment, said vector includes SEQ ID NOs: 28 and 26. In a preferred embodiment, said vector includes SEQ ID NOs: 25 and 27. In a preferred embodiment, said vector includes SEQ ID NOs: 18, 19, 20, 28 and 26. In a preferred embodiment, said vector includes SEQ ID NOs: 18, 19, 20, 25 and 27. In a preferred embodiment, said vector includes SEQ ID NOs: 12, 13, 18, 19, 20, 28 and 26. In a preferred embodiment, said vector includes SEQ ID NOs: 12, 13, 18, 19, 20, 25 and 27. In a preferred embodiment, said vector includes SEQ ID NOs: 12, 13, 28 and 26. In a preferred embodiment, said vector includes SEQ ID NOs: 12, 13, 25 and 27.

In a preferred embodiment, said vector comprises as regulatory element (i) a plant promoter from a ribulose-1,5-bisphosphate carboxylase (rubisco) small subunit and (ii) a plant terminator from a ribulose-1,5-bisphosphate carboxylase (rubisco) small subunit, wherein said regulatory elements are operably linked to the nucleic acid sequence encoding a light chain of a Cetuximab antibody, preferably SEQ ID NO: 11 and to the heavy chain of a Cetuximab antibody, preferably SEQ ID NO: 10 and the nucleic acid sequence encoding a dsRBD, preferably SEQ ID NO: 9. In a preferred embodiment, said plant promoter and said plant terminator from a rubisco small subunit of Chrysanthemum morifolium Ramat.

In a preferred embodiment, said vector includes as regulatory element a plant promoter from a rubisco small subunit of the nucleic acid sequence of SEQ ID NO: 12 and 13.

(Nucleic acid sequence of a plant promoter from a
rubisco small subunit):
SEQ ID NO: 12
AATTCGATATCACGCTTAGACAAACACCCCTTGTTATACAAAGAATTTCG
CTTTACAAAATCAAATTCGAGAAAATAATATATGCACTAAATAAGATCAT
TCCGATCCAATCTAACCAATTACGATACGCTTTGGGTACACTTGATTTTT
GTTTCAGTAGTTACATATATCTTGTTTTATATGCTATCTTTAAGGATCTT
CACTCAAAGACTATTTGTTGATGTTCTTGATGGGGCTCGGAAGATTTGAT
ATGATACACTCTAATCTTTAGGAGATACCAGCCAGGATTATATTCAGTAA
GACAATCAAATTTTACGTGTTCAAACTCGTTATCTTTTCATTTAATGGAT
GAGCCAGAATCTCTATAGAATGATTGCAATCGAGAATATGTTCGGCCGAT
ATCCCTTTGTTGGCTTCAATATTCTACATATCACACAAGAATCGACCGTA
TTGTACCCTCTTTCCATAAAGGAACACACAGTATGCAGATGCTTTTTTCC
CACATGCAGTAACATAGGTATTCAAAAATGGCTAAAAGAAGTTGGATAAC
AAATTGACAACTATTTCCATTTCTGTTATATAAATTTCACAACACACAAA
AGCCCGTAATCAAGAGTCTGCCCATGTACGAAATAACTTCTATTATTTGG
TATTGGGCCTAAGCCCAGCTCAGAGTACGTGGGGGTACCACATATAGGAA
GGTAACAAAATACTGCAAGATAGCCCCATAACGTACCAGCCTCTCCTTAC
CACGAAGAGATAAGATATAAGACCCACCCTGCCACGTGTCACATCGTCAT
GGTGGTTAATGATAAGGGATTACATCCTTCTATGTTTGTGGACATGATGC
ATGTAATGTCATGAGCCACATGATCCAATGGCCACAGGAACGTAAGAATG
TAGATAGATTTGATTTTGTCCGTTAGATAGCAAACAACATTATAAAAGGT
GTGTATCAATACGAACTAATTCACTCATTGGATTCATAGAAGTCCATTCC
TCCTAAGTATCTAAAC
(Nucleic acid sequence of a plant terminator from
a rubisco small subunit):
SEQ ID NO: 13
GGCCGCATAAGTTTTACTATTTACCAAGACTTTTGAATATTAACCTTCTT
GTAACGAGTCGGTTAAATTTGATTGTTTAGGGTTTTGTATTATTTTTTTT
TGGTCTTTTAATTCATCACTTTAATTCCCTAATTGTCTGTTCATTTCGTT
GTTTGTTTCCGGATCGATAATGAAATGTAAGAGATATCATATATAAATAA
TAAATTGTCGTTTCATATTTGCAATCTTTTTTTACAAACCTTTAATTAAT
TGTATGTATGACATTTTCTTCTTGTTATATTAGGGGGAAATAATGTTAAA
TAAAAGTACAAAATAAACTACAGTACATCGTACTGAATAAATTACCTAGC
CAAAAAGTACACCTTTCCATATACTTCCTACATGAAGGCATTTTCAACAT
TTTCAAATAAGGAATGCTACAACCGCATAATAACATCCACAAATTTTTTT
ATAAAATAACATGTCAGACAGTGATTGAAAGATTTTATTATAGTTTCGTT
ATCTTCTTTTCTCATTAAGCGAATCACTACCTAACACGTCATTTTGTGAA
ATATTTTTTGAATGTTTTTATATAGTTGTAGCATTCCTCTTTTCAAATTA
GGGTTTGTTTGAGATAGCATTTCAGCCGGTTCATACAACTTAAAAGCATA
CTCTAATGCTGGAAAAAAGACTAAAAAATCTTGTAAGTTAGCGCAGAATA
TTGACCCAAATTATATACACACATGACCCCATATAGAGACTAATTACACT
TTTAACCACTAATAATTATTACTGTATTATAACATCTACTAATTAAACTT
GTGAGTTTTTGCTAGAATTATTATCATATATACTAAAAGGCAGGAACGCA
AACATTGCCCCGGTACTGTAGCAACTACGGTAGACGCATTAATTGTCTAT
AGTGGACGCATTAATTAACCAAAACCGCCTCTTTCCCCTTCTTCTTGAAG
CTGGAGCTCG

In a preferred embodiment, said vector further comprises at least one nucleotide sequence encoding a signal peptide (signal nucleotide sequence), wherein said signal peptide targets the expressed Cetuximab antibody chain to a cell compartment, provided that said cell compartment is not the endoplasmic reticulum (ER). In a preferred embodiment, said signal peptide is an N terminal signal peptide. In a preferred embodiment, said signal nucleotide sequence is located at the 5′ end of the encoding sequence of the heavy chain and of the light chain of the Cetuximab antibody.

In a further preferred embodiment, said signal nucleotide sequence is located at the 5′ end of the encoding sequence of the heavy chain and said signal nucleotide sequence is located at the 5′ end of the encoding sequence of the light chain, with said dsRBD attached 3′ to the heavy chain.

In a more preferred embodiment, said signal nucleotide sequence is located at the 5′ end of the encoding sequence of the heavy chain and said signal nucleotide sequence is located at the 5′ end of the encoding sequence of the dsRBD, with said dsRBD attached 5′ to the light chain.

In a preferred embodiment, said at least one signal nucleotide sequence included in the vector is more than one signal nucleotide sequence which are different from each other. In a preferred embodiment, said at least one signal nucleotide sequence included in the vector is a cotton signal peptide sequence and a tobacco signal peptide sequence. This has the effect to avoid exact repeats and minimize homologous recombination and rearrangements in the vector.

More preferably, said first and second apoplast peptide are different endoglucanases. Again more preferably said first and second apoplast peptide are different endoglucanases from different species. Again more preferably said first and second apoplast peptide are different endoglucanases, wherein the first is from cotton and the second is from tobacco. Again more preferably said first and second apoplast peptide are different endoglucanases, wherein the first is of SEQ ID NO: 16 and the second is of SEQ ID NO: 17. Again more preferably said first and second apoplast peptide are different endoglucanases, wherein the first is of SEQ ID NO: 16 attached to the heavy chain and the second is of SEQ ID NO: 17 attached to the light chain.

In a preferred embodiment, said signal peptide targets the expressed Cetuximab antibody sequences to a cell compartment selected from the group consisting of cytoplasm, apoplast and vacuole. In a more preferred embodiment, said signal peptide targets the expressed Cetuximab antibody sequences to the apoplast, i.e. said signal peptide is an apoplast signal peptide. The apoplast is preferably defined as the space outside of the plasma membrane of the plant cell. In a preferred embodiment, each of said at least one signal peptide is an apoplast signal peptide.

In a preferred embodiment, said apoplast signal peptide is a human or plant apoplast signal peptide. In another preferred embodiment, said apoplast signal peptide is a plant apoplast peptide. In another preferred embodiment, said apoplast signal peptide is selected from the group consisting of an apoplast signal peptide from human, Gossypium and Nicotiana. Preferably said apoplast signal peptide is from Gossypium or Nicotiana, more preferably from Nicotiana tabacco.

In a preferred embodiment said apoplast signal peptide is an endoglucanase.

In another preferred embodiment, said at least one nucleotide sequence encoding a signal peptide has the nucleotide sequence of SEQ ID NO: 14 or 15. In a further preferred embodiment, said vector comprises one nucleotide sequence encoded by SEQ ID NO: 14 and one nucleotide sequence encoded by SEQ ID NO: 15.

(Nucleic acid sequence encoding a cotton apoplast
signal peptide):
SEQ ID NO: 14
ATGGCTAGGAAGTCCCTTATTTTCCCAGTGATCCTTCTCGCCGTGCTCCT
TTTTTCTCCACCAATCTACTCTGCTGGCCACGATTACAGGGATGCTCTCC
GTAAATCTTCCATGGCT
(Amino acid sequence encoding a cotton apoplast
signal peptide):
SEQ ID NO: 16
MARKSLIFPVILLAVLLFSPPIYSAGHDYRDALRKSSMA
(Nucleic acid sequence of a tobacco apoplast
signal peptide):
SEQ ID NO: 15
ATGGCCGCTAGGAAGTCCCTTATTTTCCCAGTGATTCTCCTCGCCGTGCT
CCTTTTTTCTCCACCAATCTACTCC
(Amino acid sequence of the tobacco apoplast
signal peptide):
SEQ ID NO: 17
MAARKSLIFPVILLAVLLFSPPIYS

In a preferred embodiment, said vector of the invention further comprises least one matrix attachment region (MAR). Said MAR is preferably selected from the group consisting of CHN 50 S/M I, CHN 50 S/M II, TM6 and Rb7.

In a preferred embodiment, said vector of the invention comprises more than one matrix attachment region (MAR) from which at least two, preferably at least three are different from each other. In a further preferred embodiment, said vector of the invention comprises CHN 50 S/M II, TM6 and Rb7. In another preferred embodiment, said vector of the invention comprises two MARs of CHN 50 S/M II, one TM6 and one Rb7.

In another preferred embodiment, said MAR is selected from the group consisting of a sequence of SEQ ID NO: 18, 19 and 20.

(Nucleic acid sequence of CHN 50 S/M II):
SEQ ID NO: 18
AGGTACAGAACGTGGGAATCTAAGTTTCTGACTCTACATTTCTACATATT
TTTAACTTCTAACTCTGAAAGCTCTTATTATACTAAATTGTGTAATTCCT
TAGTAATATGTAAATTTACTTGAACTTCTTCCAGAACCACTCCCCCAACC
TAATTATAACTTTCTAGCTAAACTCAGCGATTTTTTTGGTTCATCGTAAG
ACATTGTCAGTCGAAATATTGTACTATATCCATGTGAGGCTGATTCTTTT
TAGGAGGAGGACCTAACTCACTCAAGAGACGCCGGGTGTAACCAGGCTCT
GTTTTTTCGCCAAAACAAAAAAACTGGGAATCAAACTTTCGTGCTGCACG
TAGATATTCGCCATCTTTAAGATTAAATTGAAAACCTTCTCCTTTTTTAT
GAAATTCGTACTTAAATTTTAAAAACTCGCTTGGGCGTCATTCTGGGTGA
AATTCTTTCTTCCTCTGACTAAATTACAATTTTTTTCTAAGTAGAATGCG
TGTTCAAACATCAATTCGAACTCAAAAAATTACTTTTCTACATAGTTTAA
GAATTTCTTCAGTTCAAGTAACTAGATCTACATCCAAACACTCACCAAGT
GGGCGCTTGGCTATAAGAATTGCAAGATCCCCAAAAAGTAATAGAAACTC
TTTTCGTCGGGTAATGATATTTAGAAACTAGAATTATGCCTCGACATGAA
CATGATTTTAGGCTGTTTTTAATCGTTTGTCTCTAACCTAAACGAAAACT
TTGAAAAGTAGCTCTTTGGAGTTTTTCAAAATTTTAAGAAATTCCCAAAA
TACATTTTCAAGTAGAAGTTAAAAATACTCTAAACCAATGTCGAT
(Nucleic acid sequence of TM6):
SEQ ID NO: 19
TAATATTTAGAAATTTAATTAACATAACCAAGGATTTTTATATCGGTAAT
AACTCTAATATGGTATCCAAATCAGTCTAGAACTCTCTTACCTCTAATAA
GTAAAAGTACTTCTAATAAATTCATATACTTTTTCTCTCTTCTCCGATCT
CTCTTTGCTCTTCTTTTTATGTATCCTTTCCTTTCTAATAGCCTTTTATG
AGAAGTAAACTTTTAGGGTTGGCCCCCCCTCCCCCCACAATTATATAGTT
TCTTACTCAGTTGTTGGAATATAATTCAAATTCTTAAATAATTGACGGTG
ACATTGAGTTTTACTTTGTGGAAGAGAATTAGATTCTCGTGTTAGTAAAA
TCGGTTAGTAATTGATGATGCATTATTTTTACTCTATAATAGAGATGCAA
TTTTATTTTTGCATTTTGGGATCAAATTGTAATGCAGTCATATATTGATT
TCATAAATGTTTGGGATATTGTTGGTTATTTAACTAGAAATAGACTTCTT
ATTTCATATTTATTGTTAAAATCCTTTATTGGAGATGAATTATTTGTTCA
CCGATTAGAAGTTGATAGTCGCTTTTGTTTTAGAAGAAATTTTACCGTAG
ACCAAGTTAAGGAGTTTTAGAAGCACTTTGCATGGGAGCATTAGTGTATG
TTATGGCTTTATCAAATATAGGTTTTGAAGATTCAGAGAGCCAAGAAAAG
CTAGAACCCAAGAACTAGGAAGTTAGAGTAATTCACAATACCATAACGTG
ATATAAAACTTTTTATTGTAACTCAAATCGGTAATATTTTTTGCTTTAGT
CTTAATCGATAAATTATTTTTTTATATTGATTAGTTATAGGAGGCTCACA
AAGTTGGGAATAATTAAAATATCATATTTTGTATTTGAACAATTTATGAA
ATAGTAATTGGTAAAAAATCACTTTAAATTTTTATCCTATATCCAGAAGG
ATTATGGTGTCTGGCATAGTTGTTTGGAAGATTTGAATCAGGGTAAAAGT
ATGTTGTAATTTTTATTTTGTTATAGGCATTTTTTGTGCTTGATTGTTTT
GTTGTCATTATATTTTATTATTTGGAAGTGTATATATATGTTTGATTAAA
ATATAGATAATCAATTTTATAAGAAATTTGCAACAATTACACAAGGATAA
AGTCTACAATATGCGAGTAAAATTTGATTGAACCTAGGATGTC
(Nucleic acid sequence of Rb7):
SEQ ID NO: 20
TCGATTAAAAATCCCAATTATATTTGGTCTAATTTAGTTTGGTATTGAGT
AAAACAAATTCGAACCAAACCAAAATATAAATATATAGTTTTTATATATA
TGCCTTTAAGACTTTTTATAGAATTTTCTTTAAAAAATATCTAGAAATAT
TTGCGACTCTTCTGGCATGTAATATTTCGTTAAATATGAAGTGCTCCATT
TTTATTAACTTTAAATAATTGGTTGTACGATCACTTTCTTATCAAGTGTT
ACTAAAATGCGTCAATCTCTTTGTTCTTCCATATTCATATGTCAAAATCT
ATCAAAATTCTTATATATCTTTTTCGAATTTGAAGTGAAATTTCGATAAT
TTAAAATTAAATAGAACATATCATTATTTAGGTATCATATTGATTTTTAT
ACTTAATTACTAAATTTGGTTAACTTTGAAAGTGTACATCAACGAAAAAT
TAGTCAAACGACTAAAATAAATAAATATCATGTGTTATTAAGAAAATTCT
CCTATAAGAATATTTTAATAGATCATATGTTTGTAAAAAAAATTAATTTT
TACTAACACATATATTTACTTATCAAAAATTTGACAAAGTAAGATTAAAA
TAATATTCATCTAACAAAAAAAAAACCAGAAAATGCTGAAAACCCGGCAA
AACCGAACCAATCCAAACCGATATAGTTGGTTTGGTTTGATTTTGATATA
AACCGAACCAACTCGGTCCATTTGCACCCCTAATCATAATAGCTTTAATA
TTTCAAGATATTATTAAGTTAACGTTGTCAATATCCTGGAAATTTTGCAA
AATGAATCAAGCCTATATGGCTGTAATATGAATTTAAAAGCAGCTCGATG
TGGTGGTAATATGTAATTTACTTGATTCTAAAAAAATATCCCAAGTATTA
ATAATTTCTGCTAGGAAGAAGGTTAGCTACGATTTACAGCAAAGCCAGAA
TACAAAGAACCATAAAGTGATTGAAGCTCGAAATATACGAAGGAACAAAT
ATTTTTAAAAAAATACGCAATGACTTGGAACAAAAGAAAGTGATATATTT
TTTGTTCTTAAACAAGCATCCCCTCTAAAGAATGGCAGTTTTCCTTTGCA
TGTAACTATTATGCTCCCTTCGTTACAAAAATTTTGGACTACTATTGGGA
ACTTCTTCTGAAAATAGT

In a preferred embodiment, said nucleic acid sequence of the vector encoding the heavy and light chain of the Cetuximab antibody are each operably connected with a promoter and a terminator, both from a rubisco small subunit as regulatory elements; said promoter is flanked upstream by a MAR, and said terminator is flanked downstream by a MAR; said MAR are preferably selected from the group consisting of CHN 50 S/M I, CHN 50 S/M II, TM6 and Rb7.

In a preferred embodiment, said vector of the invention comprises from 5′ to 3′:

(1) a MAR selected from the group consisting of CHN 50 S/M I, CHN 50 S/M II, TM6 and Rb7;
(2) a first regulatory element, wherein said regulatory element is a rubisco small subunit promoter;
(3) a nucleotide sequence encoding an apoplast signal peptide, preferably from Gossypium or Nicotiana;
(4) a nucleic acid sequence encoding a first heavy or light chain of a Cetuximab antibody and a nucleic acid sequence encoding a dsRBD, wherein said first chain is (4.1) a heavy chain linked at its 3′ end (downstream) with the dsRBD encoding sequence or (4.2) a light chain linked at its 5′ end (upstream) with the dsRBD; and a nucleic acid sequence encoding second chain of a Cetuximab antibody, wherein said second chain is the other of the heavy or light chain of the Cetuximab antibody;
(5) a second of said at least one regulatory element, wherein said regulatory element is a rubisco small subunit terminator; and
(6) a second of said at least one MAR selected from the group consisting of CHN 50 S/M I, CHN 50 S/M II, TM6 and Rb7.
In a preferred embodiment, the vector of the invention comprises from 5′ to 3′ (i.e. in downstream direction): (1) a first MAR, wherein said first MAR is preferably selected from the group consisting of CHN 50 S/M I, CHN 50 S/M II, TM6 and Rb7; (2) a regulatory elements defined as a first promoter, wherein said first promoter is preferably a rubisco small subunit promoter; (3) a signal sequence encoding an apoplast signal peptide, preferably of Gossypium or Nicotiana; (4) one of a sequence encoding a heavy chain or a light chain of a Cetuximab antibody, wherein said heavy chain is linked at its 3′ end (downstream) with the dsRBD encoding sequence and the light chain is linked at its 5′ end (upstream) with the dsRBD; (5) a further regulatory element defined as a first terminator, wherein said first terminator is preferably a rubisco small subunit terminator; and (6) a second MAR, wherein said second MAR is preferably TM6 or Rb7; (7) a further regulatory elements defined as a second promoter, wherein said second promoter is preferably a rubisco small subunit promoter; (8) a signal sequence, which is encoding an apoplast signal peptide, preferably of Gossypium or Nicotiana; (9) the other of a sequence encoding either a heavy or light chain of a Cetuximab antibody, (as compared to (4)); (10) a further regulatory element defined as a second terminator, wherein said second terminator is preferably a rubisco small subunit terminator; and (11) a third MAR, wherein said third MAR is preferably selected from the group consisting of CHN 50 S/M I, CHN 50 S/M II, TM6 and Rb7.

In a preferred embodiment, said vector further comprises at least one marker cassette comprising a marker gene and at least one regulatory element for expression in plant cells, wherein said at least one regulatory element is operably linked to said marker gene. In a preferred embodiment, said marker gene is a fluorescent protein, excluding green fluorescent proteins, or an antibiotic resistance gene. Preferably said at least one regulatory element for expression in plant cells is a plant promoter and a plant terminator. In a preferred embodiment said plant promoter of the marker cassette is a CaMV 35S promoter and a NOS terminator. A preferred marker cassette comprises a red fluorescent marker gene, such as DsRed gene, a CaMV 35S promoter preferably flanked by a MAR and NOS terminator preferably flanked by a MAR, wherein said MAR are preferably selected from the group consisting of CHN 50 S/M I, CHN 50 S/M II, TM6 and Rb7. Said marker gene expressed from the marker cassette is useful as selective marker for screening transformed cells.

In a preferred embodiment, said vector further comprises a first marker cassette comprising a fluorescent protein, such as DSRed, and a second marker cassette comprising an antibiotic resistance gene, such as a Kanamycin resistance gene; each marker cassette comprises at least one regulatory element for expression in plant cells, wherein each of said at least one regulatory element is operably linked to the marker gene of its marker cassette. Preferably said at least one regulatory element for expression in plant cells is a plant promoter flanked upstream by a MAR and plant terminator flanked downstream by a MAR, wherein said MAR is preferably selected from the group consisting of CHN 50 S/M I, CHN 50 S/M II, TM6 and Rb7.

In a preferred embodiment, said marker cassette comprises a sequence of the following SEQ ID NO: 21:

TGAGACTTTTCAACAAAGGGTAATATCGGGAAACCTCCTCGGATTCCATT
GCCCAGCTATCTGTCACTTCATCAAAAGGACAGTAGAAAAGGAAGGTGGC
ACCTACAAATGCCATCATTGCGATAAAGGAAAGGCTATCGTTCAAGATGC
CTCTGCCGACAGTGGTCCCAAAGATGGACCCCCACCCACGAGGAGCATCG
TGGAAAAAGAAGACGTTCCAACCACGTCTTCAAAGCAAGTGGATTGATGT
GATATCTCCACTGACGTAAGGGATGACGCACAATCCCACTATCCTTCGCA
AGACCCTTCCTCTATATAAGGAAGTTCATTTCATTTGGAGAGGACTCCGG
TATTTTTACAACAATTACCACAACAAAACAAACAACAAACAACATTACAA
TTTACTATTCTAGTCGAAATGGCCTCCTCCGAGAACGTGATCACTGAGTT
CATGAGGTTCAAGGTGAGGATGGAAGGTACTGTGAACGGACACGAGTTCG
AGATCGAAGGTGAAGGTGAGGGTAGACCATACGAGGGACACAACACTGTG
AAGCTCAAGGTGACAAAAGGTGGCCCACTTCCATTCGCTTGGGATATCCT
TTCACCACAGTTCCAGTACGGCTCCAAGGTTTACGTTAAGCACCCAGCTG
ATATCCCCGACTACAAGAAGTTGTCTTTCCCAGAGGGATTCAAGTGGGAG
CGTGTGATGAATTTCGAGGATGGTGGTGTGGCTACTGTGACCCAGGATTC
TTCACTTCAGGATGGCTGCTTCATCTACAAGGTGAAGTTCATCGGGGTGA
ACTTCCCATCTGATGGCCCAGTGATGCAGAAAAAGACTATGGGATGGGAA
GCCTCCACTGAGAGGCTTTATCCAAGAGATGGTGTGCTCAAGGGCGAGAC
TCACAAGGCTCTTAAGCTCAAAGATGGTGGCCACTACCTCGTCGAGTTCA
AGTCTATCTACATGGCCAAGAAGCCAGTTCAGCTCCCCGGTTACTACTAC
GTTGACGCTAAGCTCGACATCACCAGCCACAACGAGGATTACACTATCGT
CGAGCAGTACGAGAGGACTGAAGGTAGGCATCACTTGTTCCTCTGAGCTT
GGAATGGATCTTCGATCCCGATCGTTCAAACATTTGGCAATAAAGTTTCT
TAAGATTGAATCCTGTTGCCGGTCTTGCGACGATTATCATATAATTTCTG
TTGAATTACGTTAAGCATGTAATAATTAACATGTAATGCATGACGTTATT
TATGAGATGGGTTTTTATGATTAGAGTCCCGCAATTATACATTTAATACG
CGATAGAAAACAAAATATAGCGCGCAAACTAGGATAAATTATCGCGCGCG
GTGTCATCTATGTTACTAGATCGGGAATTGCCAAGCTAATTCTTGAAGAC
GAAAGGGCCTCGTGATACGCCTATTTTTATAGGTTAATGTCATGATAATA
ATGGTTTCTTAGACGTGAGGTGGCACTTTTCGGGGAAATGTGCGCGGAAC
CCCTATTTGTTTATTTTTCTAAATACAGAATTGTCGACTTGTGTCGACAG
GTACAGAACGTGGGAATCTAAGTTTCTGACTCTACATTTCTACATATTTT
TAACTTCTAACTCTGAAAGCTCTTATTATACTAAATTGTGTAATTCCTTA
GTAATATGTAAATTTACTTGAACTTCTTCCAGAACCACTCCCCCAACCTA
ATTATAACTTTCTAGCTAAACTCAGCGATTTTTTTGGTTCATCGTAAGAC
ATTGTCAGTCGAAATATTGTACTATATCCATGTGAGGCTGATTCTTTTTA
GGAGGAGGACCTAACTCACTCAAGAGACGCCGGGTGTAACCAGGCTCTGT
TTTTTCGCCAAAACAAAAAAACTGGGAATCAAACTTTCGTGCTGCACGTA
GATATTCGCCATCTTTAAGATTAAATTGAAAACCTTCTCCTTTTTTATGA
AATTCGTACTTAAATTTTAAAAACTCGCTTGGGCGTCATTCTGGGTGAAA
TTCTTTCTTCCTCTGACTAAATTACAATTTTTTTCTAAGTAGAATGCGTG
TTCAAACATCAATTCGAACTCAAAAAATTACTTTTCTACATAGTTTAAGA
ATTTCTTCAGTTCAAGTAACTAGATCTACATCCAAACACTCACCAAGTGG
GCGCTTGGCTATAAGAATTGCAAGATCCCCAAAAAGTAATAGAAACTCTT
TTCGTCGGGTAATGATATTTAGAAACTAGAATTATGCCTCGACATGAACA
TGATTTTAGGCTGTTTTTAATCGTTTGTCTCTAACCTAAACGAAAACTTT
GAAAAGTAGCTCTTTGGAGTTTTTCAAAATTTTAAGAAATTCCCAAAATA
CATTTTCAAGTAGAAGTTAAAAATACTCTAAACCAATGTCGAT

Preferably, SEQ ID NO: 21 is flanked downstream and upstream by a MAR selected from the group consisting of CHN 50 S/M I, CHN 50 S/M II, TM6 and Rb7.

In a further aspect, the invention relates to a plant cell comprising the vector of the invention, wherein said plant cell is from the genus Nicotiana. In a more preferred embodiment, said plant cell is from Nicotiana tabacum or Nicotiana benthamiana. Most preferably of said plant cell is from Nicotiana tabacum. In an again more preferred embodiment, said plant cell is from Nicotiana tabacum cv. Samsun.

In a further aspect, the invention relates to a method for manufacturing a Cetuximab antibody comprising the steps of:
expressing the at least one nucleic acid sequence encoding the (heavy and/or light) chain of the Cetuximab antibody of the vector of the invention in plant cells;
optionally, screening for plant cells expressing the chain of the Cetuximab antibody; and
extracting and purifying the expressed chain of the Cetuximab antibody from the plant cells.

In a preferred embodiment, said plant cell is from the genus Nicotiana. In another preferred embodiment, said plant cell is from Nicotiana tabacum or Nicotiana benthamiana. More preferably, said plant cell is from Nicotiana tabacum. In an again more preferred embodiment, said plant cell is from Nicotiana tabacum cv. Samsun.

In plant cells, Cetuximab antibodies assemble in the presence of the Cetuximab heavy and light chain. Plant cells from the genus Nicotiana are preferred for assembly, especially from Nicotiana tabacum or Nicotiana benthamiana.

In a preferred embodiment of the method of the invention said plant cells are transformed with the vector of the invention in advance of the expression step. In a preferred embodiment said vector is transformed into said plant cells, e.g., via electroporation or a calcium chloride based method.

In a preferred embodiment, said vector is transformed into said plant cells, preferably Nicotiana tabacum, via incubation with bacteria, preferably with agrobacteria, more preferably with Agrobacterium tumefaciens, again more preferably with Agrobacterium tumefaciens strain GV3101.

In a preferred embodiment, the plant cells are screened for the expressed Cetuximab antibody chains. To screen for presence of recombinant protein expression, cell samples, such as leaf samples were analyzed with Western blot.

In a further preferred embodiment, the step of purifying the expressed Cetuximab antibody the step of purifying the expressed Cetuximab antibody comprises using a protein A column protein purification, followed by column gel filtration; or using a Protein-A-Cellulose-Binding-Domain.

After protein A column protein purification, the eluted protein can be concentrated and loaded on a gel filtration column in order to remove any protein aggregates that may have formed.

In a further aspect, the invention relates to a dsRNA binding domain (dsRBD) comprising at least amino acid residues 6-79 or 96-168 of hPKR or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In a preferred embodiment, said dsRNA binding domain (dsRBD) comprises at least amino acid residues 6-79 and/or 96-168 of hPKR, wherein cysteine at positions 121 and 135 of hPKR is exchanged by a non-cysteine amino acid. Preferably said non-cysteine amino acid is an alanine derivative. Preferably said alanine derivative is selected from the group consisting of alanine, glycine, leucine, valine, 2-aminobutyric acid, norvaline, norleucine, isoleucine and allo-isoleucine. More preferably, said alanine derivative is selected from the group consisting of alanine, glycine, leucine, valine, and isoleucine. Again more preferably said alanine derivative is alanine or valine.

In a preferred embodiment, said dsRNA binding domain (dsRBD) comprises a dsRNA binding domain (dsRBD) comprising at least amino acid residues 6-79 and 96-168 of hPKR or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 6-79 or 96-169 of hPKR, or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 6-79 and 96-169 of hPKR, or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 1-169 of hPKR, or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 1-168 of hPKR, or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 1-197 of hPKR, or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

In a preferred embodiment, said dsRNA binding domain (dsRBD) comprises amino acid residues 6-79 or 96-169 of hPKR, wherein cysteine at positions 121 and 135 of hPKR is exchanged by a non-cysteine amino acid. In a preferred embodiment, said dsRNA binding domain (dsRBD) comprises amino acid residues 6-79 and 96-169 of hPKR, wherein cysteine at positions 121 and 135 of hPKR is exchanged by a non-cysteine amino acid. In a preferred embodiment, said dsRNA binding domain (dsRBD) comprises amino acid residues 1-169 of hPKR, wherein cysteine at positions 121 and 135 of hPKR is exchanged by a non-cysteine amino acid. In a preferred embodiment, said dsRNA binding domain (dsRBD) comprises amino acid residues 1-168 of hPKR, wherein cysteine at positions 121 and 135 of hPKR is exchanged by a non-cysteine amino acid. In a preferred embodiment, said dsRNA binding domain (dsRBD) comprises amino acid residues 1-197 of hPKR, wherein cysteine at positions 121 and 135 of hPKR is exchanged by a non-cysteine amino acid.

In a preferred embodiment, said dsRNA binding domain (dsRBD) comprises a dsRNA binding domain (dsRBD) comprising at least amino acid residues 6-79 and 96-168 of hPKR or a homolog thereof, wherein in said homolog amino acid amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 6-79 or 96-169 of hPKR, or a homolog thereof, wherein in said homolog amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 6-79 and 96-169 of hPKR, or a homolog thereof, wherein in said homolog amino acid residues 1-24, 39-50 and 58-69, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 1-169 of hPKR, or a homolog thereof, wherein in said homolog amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 1-168 of hPKR, or a homolog thereof, wherein in said homolog amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. In a preferred embodiment, said dsRNA binding domain (dsRBD) comprises amino acid residues 1-197 of hPKR, or a homolog thereof, wherein in said homolog amino acid residues 1-24, 39-50 and 58-69, Y101, Y133, C135, M137, A161, F131, K150 and K154 are conserved, and wherein cysteine at position 121 and 135 of said dsRBD is exchanged by a non-cysteine amino acid. Preferably at least 80%, more preferably at least 85%, again more preferably at least 90%, again more preferably at least 95%, again more preferably at least 99% of the amino acid residues in said homolog are conserved.

The invention will now be illustrated by the following non-limiting Examples.

### EXAMPLES

### Example 1—Design and Cloning

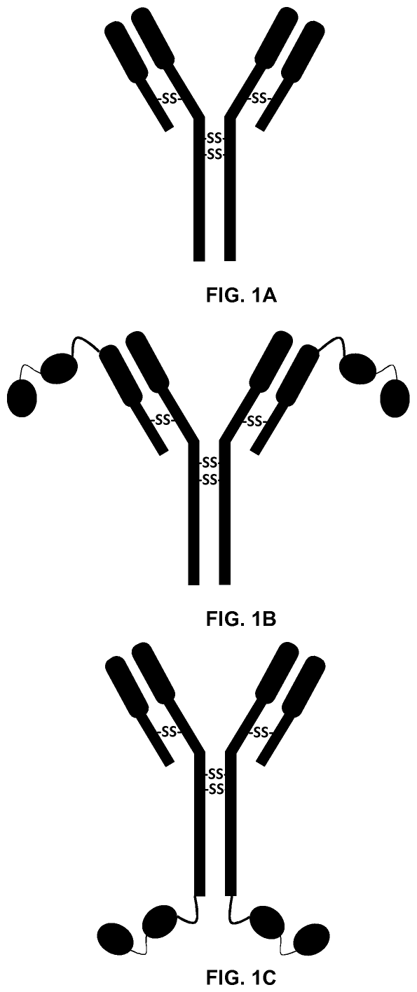

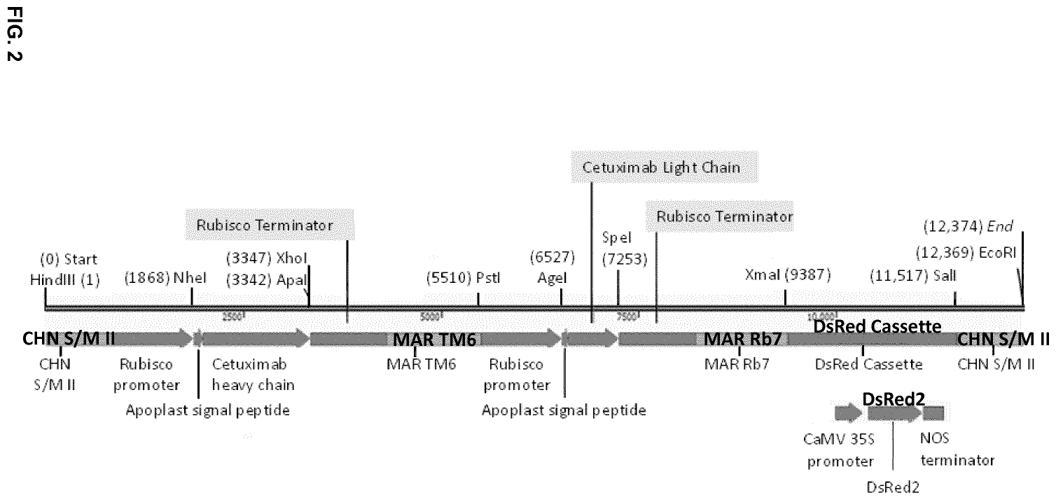

Constructs were designed for expression of the anti-EGFR antibody Cetuximab, as well as two Cetuximab-dsRBD chimeras; one with the dsRBD (SEQ ID NO: 5 and 6 or 8) bound to the light chain of the antibody at the N terminus (Cetuximab-LC-dsRBD, SEQ ID NO: 2), and one with the dsRBD bound (SEQ ID NO: 5 and 6 or 8) to the heavy chain at the C terminus (Cetuximab-HC-dsRBD, SEQ ID NO: 1) The dsRBD was bound to the antibody via a short (Gly₄Ser)₃ peptide (Cetuximab-LC-dsRBD with the linker of SEQ ID NO: 3 and Cetuximab-HC-dsRBD with the linker of SEQ ID NO: 4) (FIG. 1).

Cetuximab-DsRed:

The Cetuximab-DsRed pUC57 vector contains genes encoding the heavy chain and light chain of the Cetuximab antibody, and a gene expressing DsRed—a red fluorescent protein used as a marker for screening. These genes were codon-optimized for tobacco. The heavy and light chains were each flanked by the rubisco small subunit promoter and terminator and fused to an apoplast signal peptide (from either tobacco or cotton) directing them to the apoplast, the space outside of the plant cell plasma membrane. DsRed, a red fluorescent protein from reef coral (Discosoma spp.) was used as a marker gene and flanked by the CaMV 35S promoter and terminator. Surrounding each expression unit are four matrix attachment regions (MARs), CHN S/M II, TM6 and Rb7 (FIG. 2), which have been shown to enhance gene expression.

The recombinant anti-EGFR antibody Cetuximab and the chimeric antibody Cetuximab-dsRBD were designed using Genome Compiler and ordered from General Biosystems, Inc., North Carolina, USA, who also provided codon optimization of the sequence for tobacco. The sequences encoding Cetuximab heavy and light chains were obtained from DrugBank (https://www.drugbank.ca/drugs/DB00002).

Golden Gate Assembly Method:

The Cetuximab-DsRed pUC57 vector was assembled using the Golden Gate assembly method, which enables assembly of multiple inserts into a vector backbone in a one pot reaction using a single type IIS restriction enzyme and T4 DNA ligase. Type IIS restriction enzymes cut outside of their recognition site, leaving a 4 nucleotide overhang. Inserts can be designed with overhangs that allow assembly only in the desired order.

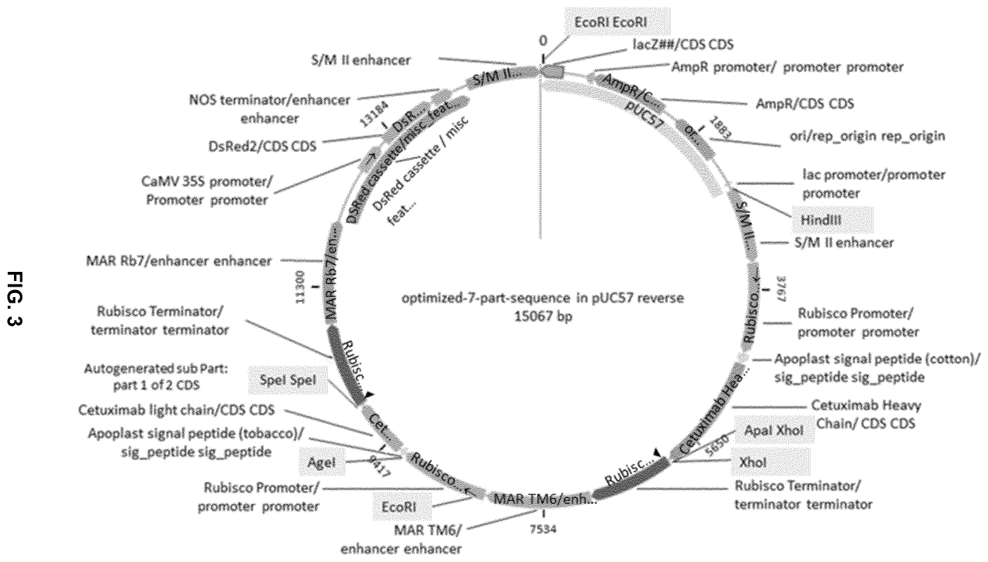

The sequence was ordered in seven parts ranging from 1-3 Kbp, and was supplied in pUC57 plasmids (FIG. 3). Each part was flanked with recognition sites for the type IIS restriction enzyme BsaI and 4 nucleotide overhangs allowing them to be assembled in the correct order using the NEB Golden Gate Assembly Tool (https://goldengate.neb.com/editor). FIG. 3 depicts the vector map of Cetuximab-DsRed in pUC57 after successful Golden Gate assembly, which was validated with DNA sequencing. Each of the seven sequence parts contained restriction sites, allowing for traditional ligation of the seven parts in case Golden Gate assembly was not successful.

The destination vector for Golden Gate assembly was prepared by inserting a short sequence containing two BsaI restriction sites in to a pUC57 vector. The four nucleotide base pair sequences on the outside of the BsaI restriction sites were complimentary to the first and last of the seven sequence parts. The Golden Gate assembly reaction was comprised of 100 ng of the destination vector, each of the seven parts at a 2:1 insert:vector molar ratio, 1.5 μl 10×NEB T4 buffer, 1.5 μl 10×BSA, 1 μl NEB T4 ligase (2,000,000 units/ml), 1 μl BsaI (10,000 units/ml) in a final volume of 15 μl. The assembly reaction was performed in a thermocycler as follows: (3 min 37°, 4 min 16°)×24, 5 min 50°, 5 min 80°, hold 16°. 5 μl of the reaction was transformed into competent E. coli, strain DH5a.

Cetuximab-dsRBD Chimeras:

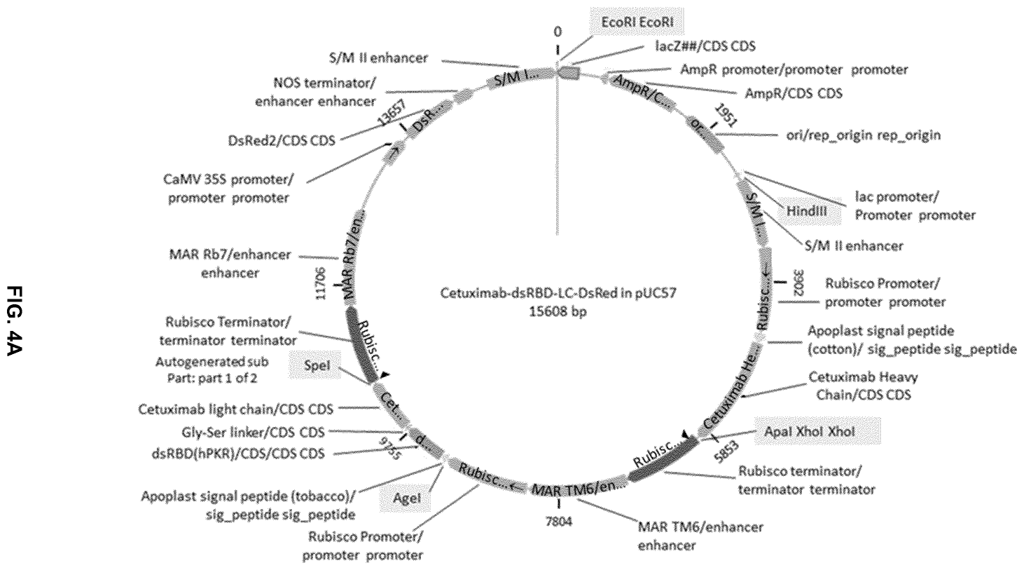

The obtained Golden Gate assembly product, Cetuximab-DsRed pUC57 vector, was further modified to create the Cetuximab-dsRBD chimeras. The vector was cut (with ApaI and XhoI) to remove the heavy chain stop codon, and then ligated with a fragment encoding a flexible protein linker (Gly₄Ser)₃ and the dsRBD, thus creating Cetuximab-Heavy Chain-dsRBD-DsRed (FIG. 4B). Similarly, the vector was cut (with AgeI and SpeI) to remove the light chain, and then ligated with a fragment encoding the dsRBD, linker and light chain, thus creating Cetuximab-dsRBD-Light Chain-DsRed (FIG. 4A).

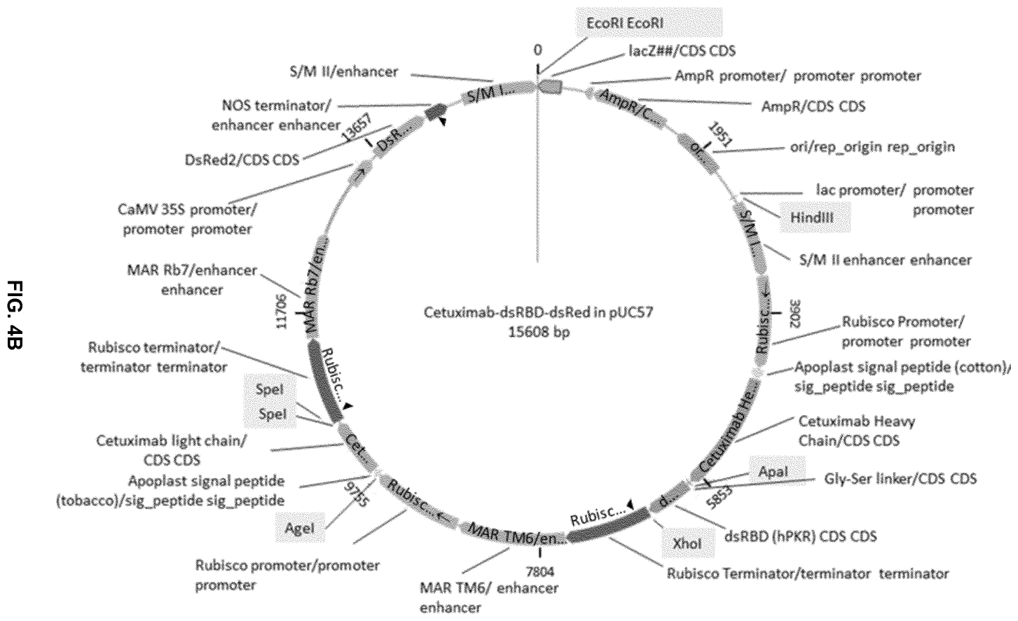

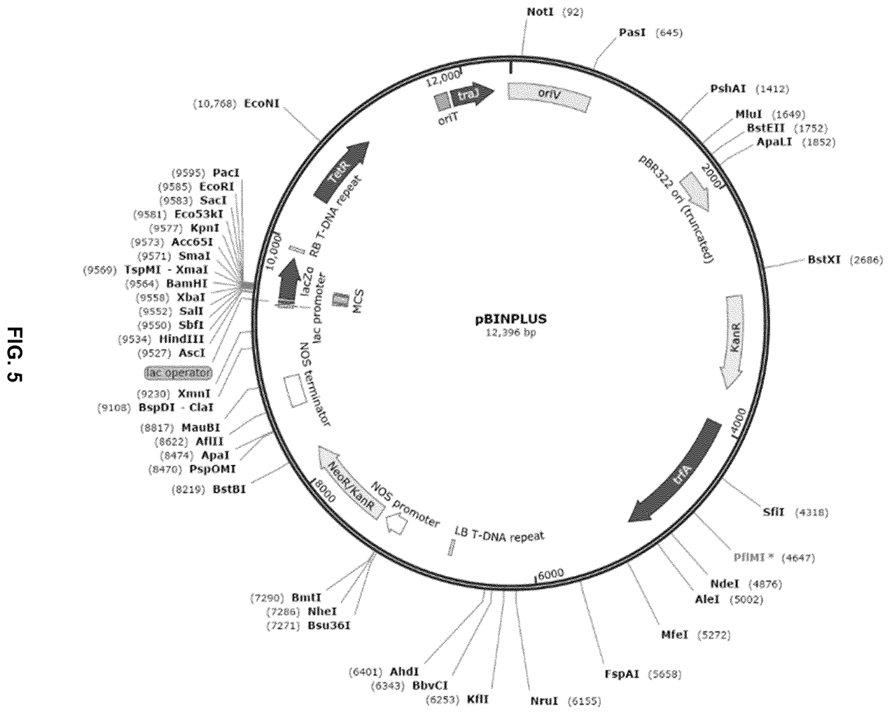

After sequence confirmation, the inserts (Cetuximab-DsRed, Cetuximab-Heavy Chain-dsRBD-DsRed and Cetuximab-dsRBD-Light Chain-DsRed) were then each cut out of the pUC57 with EcoRI and HindIII and transferred to the multiple cloning site (MCS) of pBINPLUS binary expression vectors (FIG. 5).

The expression units for Cetuximab, Cetuximab-LC-dsRBD and Cetuximab-HC-dsRBD were each separately engineered into pUC57 and each unit was then cut out of the pUC57 vector and separately inserted in the pBINPLUS vector.

The T-DNA area of pBINPLUS also contained Kanamycin resistance near the left border, which was used as a selective marker. The MCS was located on the plasmid within the T-DNA (between the left and right T-DNA borders)—the area of DNA that was transferred via agrobacterium mediated transformation to the tobacco.

### Example 2—Agrobacterium Electroporation

The pBINPLUS vectors encoding Cetuximab, Cetuximab-LC-dsRBD and Cetuximab-HC-dsRBD were then transformed into Agrobacterium tumefaciens strain GV3101 using electroporation. Due to poor efficiency of DNA isolation in agrobacterium, in order to validate successful transformation, DNA was isolated from the agrobacterium and then re-transformed into E. coli. DNA was then isolated and sequenced from the re-transformed E. coli. Agrobacterium clones with validated plasmid sequences were then used for tobacco transformation.

### Example 3—Plant Transformation

Nicotiana tabacum cv. Samsun plants were transformed via agrobacterium mediated tobacco transformation. Leaf pieces from sterile grown tobacco were incubated with the recombinant agrobacterium, and then grown on MS medium plates containing Kanamycin for about 3-5 weeks. Shoots that developed were grown on rooting medium, and plantlets were moved to the greenhouse once roots developed. Leaves were sampled soon after being moved to greenhouse, approximately 8-10 weeks post transformation.

In detail, sterile Nicotiana tabacum cv. Samsun plants were grown for 6-8 weeks. Each of the three engineered agrobacterium strains was grown in 50 ml LB with 50 μg/ml Kanamycin for approximately 48 hours, on a shaker, at 28°. The bacterial cultures were centrifuged for 10 minutes, 5000 RPM, at room temperature. The supernatant was removed and each pellet was re-suspended with 50 ml MS medium (4.4 g/L Murashige &Skoog medium including vitamins (Duchefa cat #M0222.0050) and 30 g/L sucrose (J. T. Baker cat #4072-05), pH=5.8). Tobacco leaves were cut into 1 cm² pieces and incubated for 5 minutes with the agrobacterium. Leaf pieces were then plated on Petri dishes with solid MS medium (liquid MS medium with 7 g/l plant agar (Duchefa (cat #P1001.1000)), containing 0.8 ml/L indole-3-acetic acid (IAA) and 2 ml/L kinetin. Plates were incubated in the dark at 25° for 48 hours, after which the explants were transferred to new Petri dishes containing selective MS medium (0.8 ml/L IAA, 2 mUL kinetin, 400 mg/L carbenicillin and 100 mg/L kanamycin).

Plates were incubated in the light at 25°, and moved to new selection every 10 days for 3-4 weeks. After calluses developed and shoots formed, the shoots were moved to rooting medium (MS medium with 100 mg/L kanamycin and 400 mg/L carbenicillin), until roots developed (3-4 weeks). Shoots were then transferred to soil pots in a greenhouse, connected to a drip irrigation system, and grown to mature plants.

After identification of recombinant plants (below), the plants were self-fertilized repeatedly (up to 10 times) in order to obtain homozygotes.

### Example 4—SDS-PAGE and Western Blot Analysis

Screening of Plant Lines:

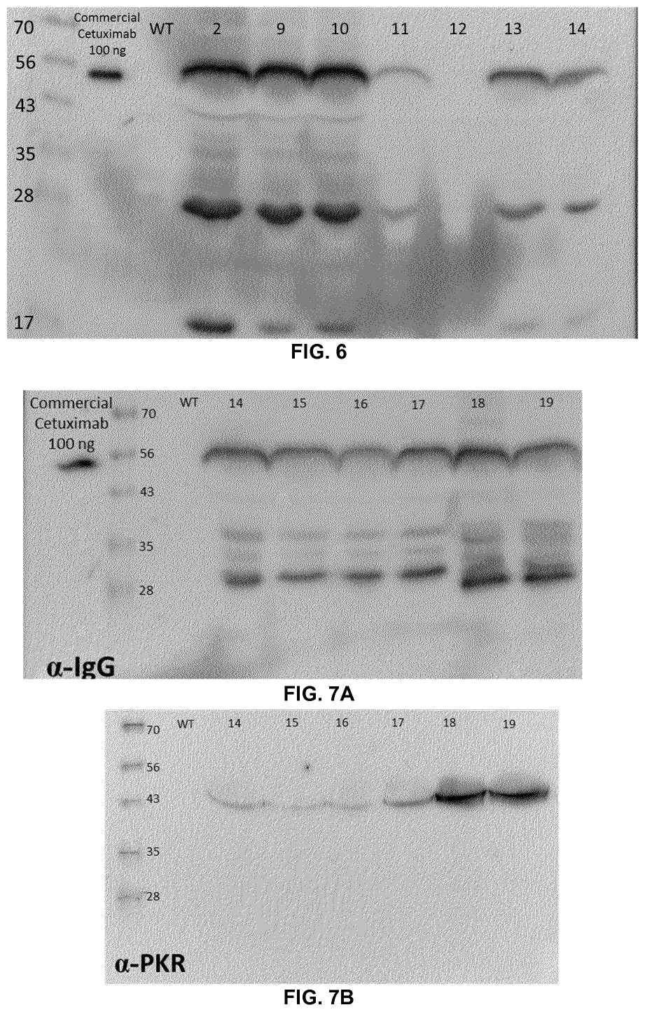

To screen for presence of recombinant protein expression, leaf samples were analyzed with Western blot using an anti-human IgG antibody. In the Cetuximab expressing plants, bands corresponding to Cetuximab heavy chain and light chain were detected at ˜50 and ˜25 kDa (FIG. 6). A small band at approximately 17 kDa was also present in all the samples, which was believed to be a degradation product of the light chain, as is known to happen when expression levels of the light chain are higher than those of the heavy chain. This was later confirmed using an anti-kappa light chain antibody.

In Cetuximab-LC-dsRBD expressing plants, the light chain was 20 kDa larger due to the addition of the PKR dsRNA binding domain. Western blots using anti-IgG antibody showed a band at ˜50 kDa, for the heavy chain, and a faint band at ˜45 kDa, the molecular weight for the light chain-dsRBD chimera. When using an anti-PKR antibody, which detected the dsRBD, the band at ˜45 kDa was more prominent. In addition to these two bands, some smaller bands were detected when using the anti-IgG antibody, including a band at ˜25 kDa, the molecular weight of the light chain without the dsRBD. These bands were all detected when using an anti-kappa antibody, suggesting that the LC-dsRBD underwent partial degradation. However, as there is a band at the correct molecular weight of the LC-dsRBD, it is confirmed that some of the expressed protein is the desired chimera (FIG. 7).

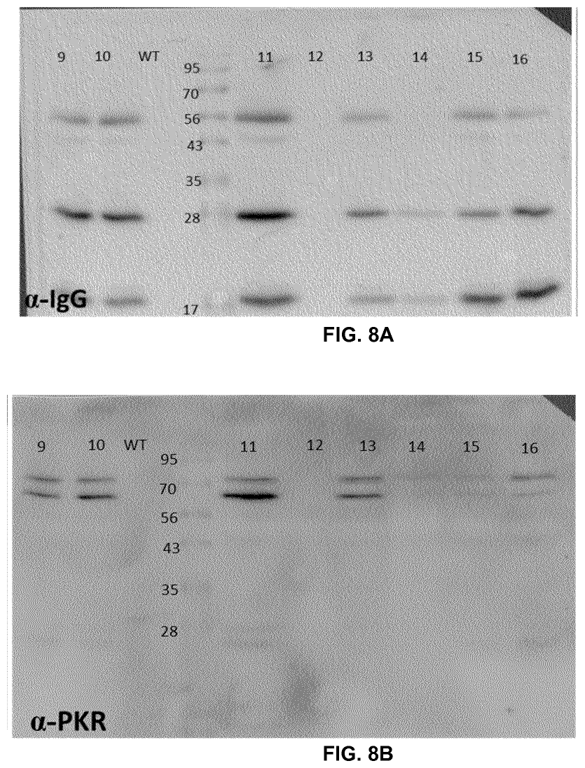

Similarly, in Cetuximab-HC-dsRBD expressing plants, the heavy chain was 20 kDa larger. When using an anti-human IgG antibody, bands were detected at ˜50 and ˜25 kDa, representing the original heavy and lights chains, but not the chimeric heavy chain-dsRBD (FIG. 8). Because of the addition of the dsRBD, the anti-IgG antibody is obscured from binding the chimeric heavy chain-dsRBD. When using an anti-PKR antibody, bands were detected at the expected molecular weight of ˜75 kDa, in addition to a smaller band of ˜65 kDa. The dsRBD of PKR is comprised of two double-stranded RNA binding domains, so the smaller band represents a chimeric heavy chain-dsRBD with only one dsRNA binding domain. Thus, the desired chimera is also present.

Plant Sample Preparation for SDS-PAGE and Western Blot Analysis:

In detail, pre-weighed microfuge tubes were prepared with 100 μl grinding buffer (100 mM Tris-HCl pH 8, 25 mM NaCl, 10 mM EDTA, 1 mM potassium metabisulfite (PMBS), 1× Complete Protease Inhibitor Tablets). Approximately 10 weeks post transformation; four leaf discs (˜80 mg) from each plant were clipped with the microfuge lid in to the tube, placed on ice and weighed. The samples were ground for 30 seconds with a plastic micro-pestle attached to an overhead stirrer. Samples were centrifuged for 30 minutes at 4000 RPM speed at 4° and supernatant was transferred to new tubes for analysis.

SDS-PAGE and Western Blot Analysis:

Prepared plant samples were run on a 12% SDS gel and transferred to a nitrocellulose membrane. The membrane was blocked with 5% skim milk in TBST for 1 hour at room temperature and then incubated with an anti-human IgG (H+L), HRP conjugated antibody (Jackson ImmunoResearch, 1:10,000) for two hours at room temperature. After washing the membrane 3 times for 5 minutes with TBST, ECL (Enhanced chemiluminescence) reagents were added and the signal was captured using an ECL documentation system. For detection of the dsRBD, a mouse derived anti-PKR primary antibody was incubated with the membrane overnight at 4°, the membrane was washed 3 times for 5 minutes with TBST and then incubated for one hour with a secondary anti-mouse HRP conjugated antibody.

### Example 5—Plant Sample Preparation for ELISA and Protein Purification

Leaves from the top, bottom and middle of 2-3 month old plants were collected (5-20 grams per plant). 10 ml of grinding buffer (100 mM Tris-HCl pH 8, 25 mM NaCl, 10 mM EDTA, 1 mM PMBS, 1× anti-protease cocktail (20 mM AEBSF, 16 mM aprotinin, 130 mM benzamidine, 2 mM leupeptin, 50 μM Soybean Trypsin Inhibitor)) was added per gram of plant tissue and blended with a Waring laboratory blender for 30 seconds. Samples were centrifuged for 30 minutes at 9000 RPM at 4°, supernatant was transferred to new tubes and centrifugation was repeated. Samples were frozen at −20°.

### Example 6—ELISA

To determine functionality, as well as to quantify the amount of protein expressed in the tobacco plants, a sandwich ELISA was performed using an EGFR coated plate. A positive reaction was observed with the expressed Cetuximab as well as the Cetuximab-LC-dsRBD. A positive reaction was also observed with the Cetuximab HC-dsRBD at a lower intensity. This lower reactivity is most likely due to the difficulty of the detection reagent, an anti-Cetuximab antibody, to bind the chimeric HC-dsRBD. The ELISA was used to assess expression levels of the four highest expressing plants of each construct, based on the Western blot results. This allowed for selection of the plant with the highest expression level, which was self-pollinated for continued growth of Cetuximab and Cetuximab-dsRBD expressing plants, as well as eventual generation of a homozygote seed bank. The highest expressing Cetuximab (CTX), Cetuximab-LC-dsRBD, and Cetuximab-HC-dsRBD plants had concentrations of 57, 45 and 14 mg/kg, respectively. It must be taken in to account that not all of the protein in the Cetuximab-LC-dsRBD sample contained the dsRBD, and that the detection reagent may not have been able to bind to all of the Cetuximab-HC-dsRBD.

A Cetuximab ELISA kit was purchased from SomruBioScience. Prepared plant samples were diluted 1:100 in assay buffer (for a final dilution of 1:1000) and 100 μl of each sample was added to EGFR coated wells. A standard curve was prepared with concentrations of Cetuximab ranging from 0.156-10 ng/well. The plate was incubated on a plate shaker at 23° for one hour at 300 RMP. Plate content was discarded and the plate was washed 3 times 3 minutes with 250 μl wash buffer. 100 μl detection reagent was added, and the plate was incubated on a plate shaker at 23° for one hour at 300 RMP. Following 3 more washes of 250 μl wash buffer, 100 μl TMB was added to the wells and the plate was incubated for 7 minutes, protected from light. 100 μl TMB stop solution was added and results were read with a plate reader at 450 nm, with a background at 620 nm. The calibration curve was calculated using the 4 parameter logistic regression (4PL) model.

In a ELISA assay specifically designed for characterizing binding ability of the recombinant protein of the invention, e.g. Cetuximab-LC-dsRBD, and Cetuximab-HC-dsRBD, maxisorp 96-well plates were coated overnight at 4° C. with 1 μg/ml R-Hum-hEGFR (Extracellular domain active EGFR AA 1-645, product no. ABIN2001843 from Antibodies Online) in 0.1 M Carbonate buffer pH 9.6. The plate was then washed four times with PBS pH 7.4 0.1% v/v Tween-20, and blocked with the same wash buffer including 2% skim milk for 2 h at 37° C. Following washing, dsRBD-LC was added, with serial 1:1 dilutions. A calibration curve with Cetuximab ranging from 0.1 μg/ml to 0.002 μg/ml was included. The plate was incubated for 45 min at 37° C. The plate was washed as above, and incubated with peroxidase-labeled anti-human IgG, diluted 1/200000 in PBS 0.1% tween-20 2% skim milk, for 30 min at 37° C. The plate was washed as above, and 100 μl of TMB solution was added to each well. The plate was incubated in the dark for 2 min at room temperature, and the reaction was stopped by adding 50 μl of 1M sulfuric acid to each well. Absorbance was recorded at 450 nm, with background correction at 620 nm. Dose-dependent binding of commercial Cetuximab as well as tobacco-expressed CTX could be shown following purification on protein A, and a crude lysate of the dsRBD-LC chimera to EGFR (cf. FIG. 12).

These data evidence that the tobacco CTX and the dsRBD-LC Cetuximab chimera are capable of binding to EGFR in a specific manner. To confirm the specificity of binding competition experiments with EGF are performed.

### Example 7—Protein a Bead Protein Purification

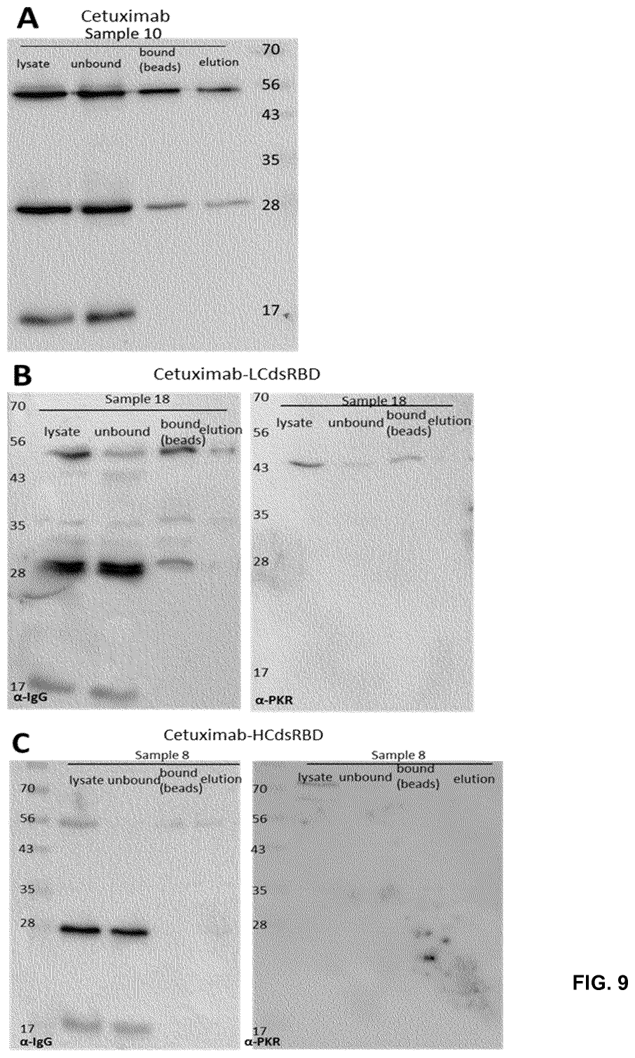

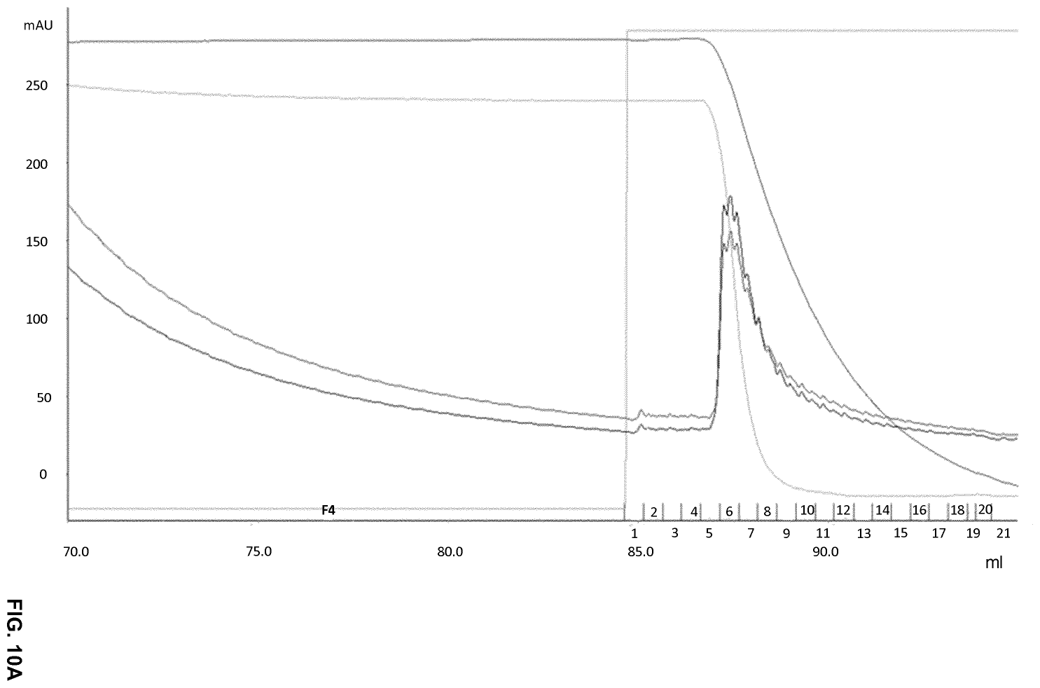

Before attempting to purify the plant derived Cetuximab and Cetuximab-dsRBD chimeras using a protein A column, a small scale purification was carried out using Protein A agarose beads to validate protein A binding. Results showed that Cetuximab and Cetuximab-LC-dsRBD bound the protein A beads at pH 7.2 and were eluted at pH 3 (FIG. 9). In addition, the 17 kDa light chain fragment that appears in the lysates before purification is not present in the elution.

Cetuximab-HC-dsRBD, however, did not seem to be able to bind to the protein A beads, probably since the addition of the dsRBD to the heavy chain obscure protein A-Fc binding. Thus, protein A column purification was carried out only with Cetuximab and Cetuximab-LC-dsRBD.

In detail, 40 μl Pierce protein A agarose beads were added to each dolphin microfuge tube, washed with 500 μl DDW, and then centrifuged for 2 minutes at 2500 g in a swinging bucket centrifuge. Wash was repeated twice with 500 μl IP buffer (25 mM Tris, 150 mM NaCl pH 7.2. 900 μl of prepared plant samples were added to beads and incubated for 2 hours at room temperature with rotation. Samples were centrifuged for 2 minutes at 2500 g and supernatant was discarded. Beads were washed 3 times with 500 μl IP buffer. 50 μl elution buffer (100 mM citric acid) was incubated with the beads for 5 minutes, followed by centrifugation for 2 minutes at 2500 g and supernatant was collected. The elution step was repeated and supernatants were combined. 37 μl 1M Tris pH 8.5 was added to elution to restore physiological pH.

### Example 8—Protein a Column Protein Purification

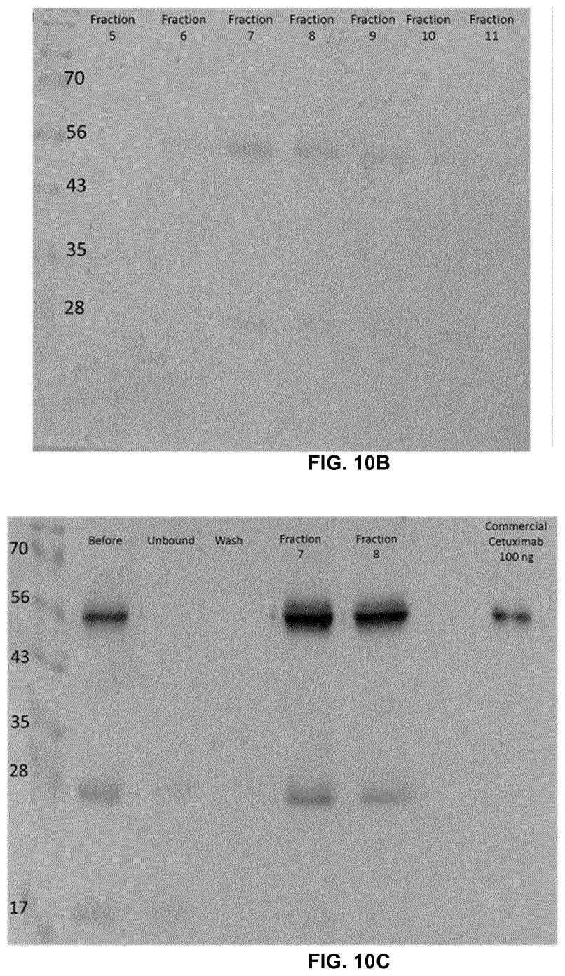

Cetuximab and Cetuximab-LC-dsRBD plant lysates from high expressing plants were purified on a protein A column using an ÄKTA design chromatography system. Elution was carried out under acidic conditions, and fractions were collected. Fractions from elution peak were run on SDS-PAGE for Coomassie staining and Western blot analysis. For Cetuximab purification, heavy and light chains were detected in the eluted fractions at ˜50 and ˜25 kDa, respectively (FIG. 10).

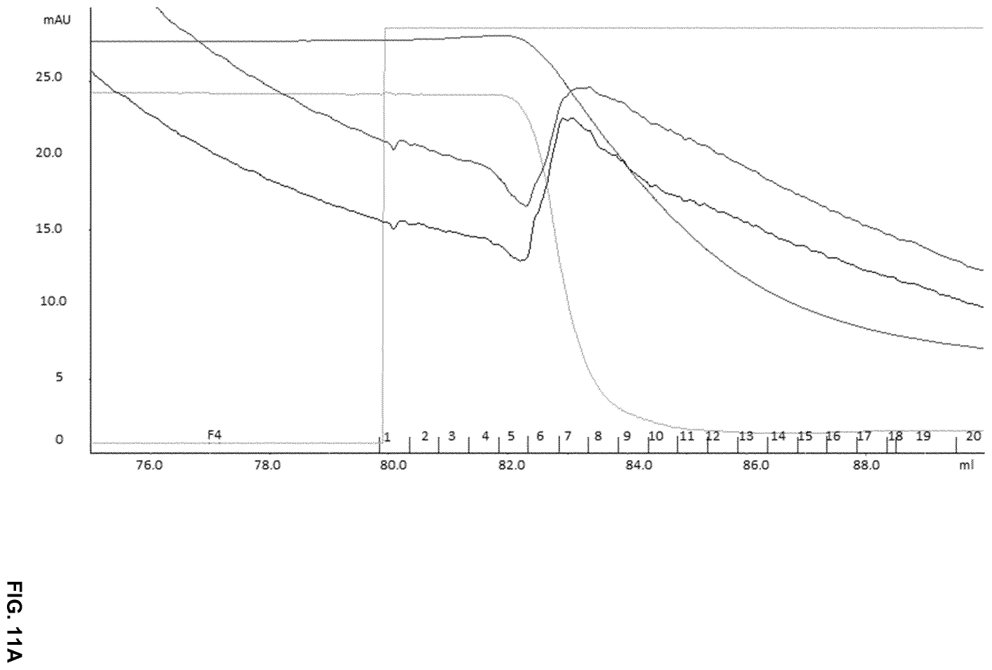

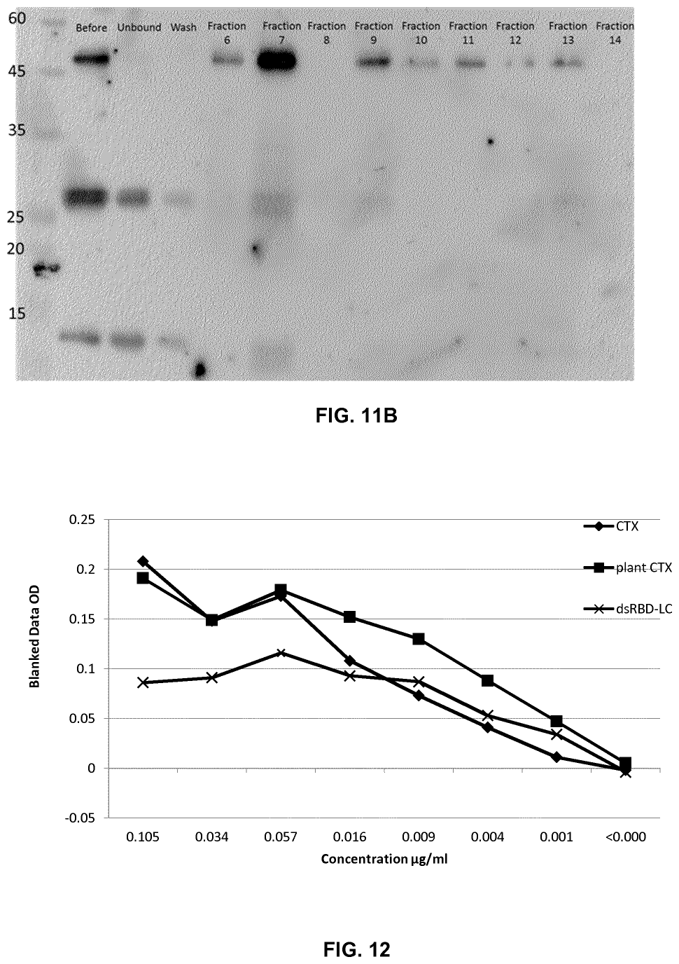

For Cetuximab-LC-dsRBD purification, there was a much smaller peak, as a smaller amount of plant tissue was used than for Cetuximab purification (FIG. 11). However, while Western blot analysis of the eluted fractions shows a clear band at ˜50 kDa, representing the heavy chain, as well as a band at ˜25 kDa that could represent the light chain without the dsRBD, there is no band representing the LC-dsRBD. Using an anti-PKR antibody to better detect the LC-dsRBD also did not result in detection of any bands (results not shown). It is possible that more plant tissue is needed in order for some of the eluted protein to contain the LC-dsRBD, or it is possible that steps in the handling process led to degradation of the LC-dsRBD. We are currently working on investigating this issue and achieving purification of Cetuximab-LC-dsRBD.

Following protein A column purification, the eluted protein was concentrated and loaded on a gel filtration column in order to remove any protein aggregates that may have formed. After collecting the protein fractions in the desired buffer, the protein was again concentrated and frozen at ˜80° C. Protein concentration was measured with a NanoDrop. The concentration of purified Cetuximab was ˜10 mg/kg and the concentration of purified Cetuximab-LC-dsRBD was ˜1 mg/kg.

In detail, prepared plant extracts were filtered through a 0.2 μm filter and passed through a HiTrap Protein A High Performance 1 ml column, using an ÄKTA chromatography system. Sodium phosphate buffer (10 mM sodium phosphate pH 7.4, 150 mM NaCl) was used to wash the column. Elution was performed with 100 mM citric acid, pH 3. Fractions were collected in tubes containing neutralization buffer (Tris pH 9.5) at the volume required to achieve a final pH of 7.4 and analyzed by SDS PAGE. Fractions that contained protein were combined, concentrated, and purified using a gel filtration column. Fractions containing protein were again combined, concentrated, and frozen at ˜80°. Protein concentration was measured using a NanoDrop.

### Example 9—Large Scale Purification

For larger scale purification, the chimeric protein Protein-A-Cellulose-Binding-Domain is used (ProtA-CBD) (E. Shpigel et al., Expression, purification and applications of staphylococcal Protein A fused to cellulose-binding domain, Biotechnol. Appl. Biochem. 2000, vol. 31(3), p. 197). In this chimeric protein, Protein A (ProtA), which binds specifically to the Fc of IgG molecules, is fused to a cellulose binding domain. Cellulose is commercially available in many forms at a very low price and is thus an attractive matrix for affinity purification. Tobacco plants are cross-bred with high yield of recombinant Cetuximab or Cetuximab-dsRBD with tobacco plants expressing ProtA-CBD. Therefore, in these crossbred plants, the expressed recombinant antibody is bound via the Fc to ProtA-CBD. After protein extraction, nitrocellulose is added to the protein extract and filtered through a 0.2 micron holofiber. The ProtA-CBD-Antibody compound binds to the nitrocellulose and is not able to pass through the holo fiber, while the other unwanted proteins pass through and are discarded. A series of pH changes and washes cause the recombinant protein to be released from the ProtA-CBD and eluted, ready for use in further experiments.

### Example 10—Assembly of the Heavy and Light Chain of the Chimeric Protein

The assembly of the heavy and light chain of the chimeric protein took place in the cells of the expression system used, e.g. in the cells of the tobacco plants.

The light chain and heavy chain are expressed separately, then form S—S bonds (both within each heavy and light chain and between the heavy and light chains), and are folded into the final structure, which is made up of 2 heavy and 2 light chains. This is a process that happens inside the cells, using the cell machinery (Feige and Buchner, Principles and engineering of antibody folding and assembly, Biochimica et Biophysica Acta (BBA) Proteins and Proteomics, 2014, vol. 1844 (11), pp. 2024-2031; Ma et al., Assembly of monoclonal antibodies with IgG1 and IgA heavy chain domains in transgenic tobacco plants, Eur. J. Immunol. 1994. 24: 131-138).

### Example 11—PolyIC Binding of the Cetuximab Chimera

To evaluate the ability of the Cetuximab chimera to bind polyIC, an electrophoretic mobility shift assay (EMSA) test is performed. Different amounts of the purified chimera are incubated with dsRNA (i.e. Cy3-labeled polyIC or dsRNA of a defined length) and electrophoresed on agarose gel. After visualization of the gel, migration of the dsRNA incubated with the chimera is compared with dsRNA without the chimera. Retarded migration shows the ability of the chimera to bind dsRNA.

### Example 12—Biological Activity of the Cetuximab Chimera

Cetuximab chimera with dsRBD attached to the N-terminus of the light chain is able to bind to EGFR. The ability of the chimera to bind EGFR was shown by ELISA, which is performed on plates coated with EGFR. EGFR attached to the plate was followed by Cetuximab or dsRBD-Cetuximab chimera, followed by peroxidase conjugated anti human IgG. Binding of the Cetuximab-dsRBD chimera with polyIC and without polyIC to EGFR was comparable to binding of commercially available Cetuximab.

To confirm that the binding is specific to EGFR, free EGF is added in increasing doses. It is tested whether EGF competes with the chimera for binding to EGFR, thus reducing the signal obtained with the peroxidase conjugated anti-IgG in the ELISA. Cytotoxicity of the chimera is demonstrated by the following assays.

Survival Assay:

EGFR over-expressing cells are plated in 96-well plates (5000 cells per well). Medium is changed the next day, and the cells are treated with polyIC, Cetuximab, Cetuximab-dsRBD chimera (“CTX-dsRBD”), or polyIC which has been pre-incubated with Cetuximab-dsRBD chimera (“polyIC/CTX-dsRBD”) at a predetermined ratio. Cell survival is measured with the methylene blue colorimetric assay 72 hours after treatment.

CTX-dsRBD, in the absence of polyIC, will be as effective as Cetuximab (leading to 20-80% survival, depending on the cell line), but polyIC/CTX-dsRBD will strongly decrease the survival of cells that are barely affected by Cetuximab, e.g. MDA-MB-468.

Cell Cycle Analysis:

EGFR over-expressing cells are plated in 6-well plates (500,000 cells per well). Medium is changed the next day, and cells are treated with polyIC, Cetuximab, CTX-dsRBD, or polyIC/CTX-dsRBD at different concentrations for 48 hours. Cells are dissociated with trypsin and centrifuged for 10 min at 500 g. Cell pellets are washed twice with PBS, and cell membranes are damaged by repeated freeze-thaw cycles in liquid nitrogen. Alternatively, cells can be fixed and permeabilized with ethanol. Cells are incubated with 0.2 ml of ribonuclease A (1 mg/ml) and stained with 0.2 ml of propidium iodide solution (100 μg/ml). Fluorescence of cells is analyzed using flow cytometry, BD FACS ARIAIII (BD Biosciences, USA). Cell cycle distributions are calculated using appropriate software. Various kits are available to simplify the procedure, e.g. BioVision's EZ Cell Cycle Analysis kit.

CTX-dsRBD, in the absence of polyIC, will arrest cells in G1, as does Cetuximab. We anticipate that polyIC/CTX-dsRBD will drive most of the cells into apoptosis, and that the remaining cells may be arrested in G1.

Apoptosis Assay:

EGFR over-expressing cells are plated in 24-well plates (100,000 cells per well). Medium is changed the next day and the cells are treated with polyIC, Cetuximab, CTX-dsRBD, or polyIC/CTX-dsRBD at a predetermined ratio for 8 hours. Annexin V/Propidium iodide (PI) staining is performed using the MBL MEBCYTO apoptosis kit according to the manufacturer's guidelines and analyzed using flow cytometry, BD FACS ARIAIII (BD Biosciences, USA).

CTX-dsRBD, in the absence of polyIC, will cause low levels of apoptosis (<20%), but polyIC/CTX-dsRBD will drive most of the cells into apoptosis.

Western Blot Analysis:

EGFR over-expressing cells are plated in 6-well plates (500,000 cells per well). Medium is changed the next day and the cells are treated with polyIC, Cetuximab-dsRBD, or polyIC which has been pre-incubated with Cetuximab-dsRBD at different concentrations. At different time points, the cells are lysed with boiling Laemmli sample buffer (10% glycerol, 50 mmol/L Tris-HCl, pH 6.8, 3% SDS, and 5% 2-mercaptoethanol). The lysates are subjected to western blot analysis with antibodies against the following: EGFR, phospho-EGFR, ERK, phospho-ERK, CDK2, phospho-p130, phospho-Rb, p27, BCL2 and Bax, caspase 3, caspase 9 and PARP.

ELISA:

EGFR over-expressing cells are plated in 96-well plates (5000 cells per well). Medium is changed the next day and the cells are treated with polyIC, Cetuximab-dsRBD, or polyIC which has been pre-incubated with Cetuximab-dsRBD at a predetermined ratio. Medium is collected after 24 hours, and Interferon gamma-induced protein 10 (IP10), chemokine (C—C motif) ligand 5 (CCLS) and tumor necrosis factor alpha (TNFα) proteins are quantified using ABTS ELISA Development Kits (PeproTech) according to the manufacturer's protocol. Interferon beta (IFN-β) protein is quantified using a bioluminescent ELISA kit (LumiKine) according to the manufacturer's protocol.

PMBC Bystander Effects:

EGFR over-expressing cells are plated in 6-well plates (500,000 cells per well). Medium is changed the next day and the cells are treated with polyIC which has been pre-incubated with Cetuximab-dsRBD at a predetermined ratio. 48 hrs after treatment, 0.5 ml of medium from the transfected cells (“conditioned medium”) is added to 500,000 PBMCs which have been seeded 24 hrs earlier into 24 well plates and grown in 0.5 ml medium. 0.1 ml of medium from the conditioned PBMCs is then exchanged for 0.1 ml medium from additional non-treated EGFR over-expressing cells (“indicator cells”) seeded on 96 well plates 24 hrs earlier. Survival of these cells is determined by methylene blue assay 48 hours after addition of medium from the PBMCs.

Direct Bystander Effects:

In parallel to previous assay, to show the direct bystander effect, 0.1 ml of conditioned medium is used to replace 0.1 ml medium from non-transfected indicator cells seeded 24 hrs earlier onto 96 well plates and grown in 0.2 ml medium. Survival of these cells is determined 48 hours after addition of the conditioned medium using methylene blue.

In Vitro Cancer Cell Killing by Activated PBMCs:

20,000 EGFR-over expressing cells are seeded onto 24 well plates and grown overnight in 1 ml RPMI medium supplemented with 10% FCS and antibiotics. Cells are then treated with polyIC which has been pre-incubated with Cetuximab-dsRBD at a predetermined concentration. 24 hrs later 500,000 PBMCs/well are added to the cancer cells and co-incubated for another 24 hrs. Apoptotic cells are visualized using an Annexin-V-Biotin kit (Biosource, Inc.). To distinguish tumor cells from PBMCs, tumor cells are labeled with FITC-conjugated EGFR antibody (Biosource, Inc., green fluorescence). Cells are visualized with a fluorescent microscope and photographed using a digital camera. Alternatively, apoptosis can be analyzed by FACS, with gating to ignore the PBMCs.

Results:

When treating EGFR-over-expressing cell lines with Cetuximab-dsRBD-polyIC, cell growth will be inhibited, and apoptosis, G1 population, p27 and Bax will be increased, whereas CDK2, phosphor-p130, phospho-Rb, phospho-EGFR, phosphor-ERK, and BCL2 will be decreased. When treating with Cetuximab-dsRBD-polyIC, cytokine and chemokine levels will be elevated, and direct as well as PBMC-mediated bystander effects will occur. Due to the synergistic effect of polyIC combined with Cetuximab, the effects induced by treatment with Cetuximab-dsRBD-polyIC will exceed the effects evoked by Cetuximab monotherapy.

1. A recombinant protein comprising a double stranded RNA (dsRNA) binding domain and a Cetuximab antibody.

2. The recombinant protein of claim 1, wherein said dsRNA binding domain (dsRBD) is bound to the N terminus of a light chain of said Cetuximab antibody, or said dsRBD is bound to the C terminus of a heavy chain of said Cetuximab antibody.

3. The recombinant protein of claim 1 or 2, wherein said dsRBD and said Cetuximab antibody are covalently bound via a spacer peptide, wherein said spacer peptide comprises the peptide (Gly₄Ser)ₙ, wherein preferably n is 1, 2, 3 or 4, further preferably n is 3.

4. The recombinant protein of claim 3, wherein said dsRBD is bound to the N terminus of the light chain of said Cetuximab antibody via said spacer peptide, wherein said spacer peptide has an amino acid sequence of SEQ ID NO: 3 (GGGGSGGGGSGGGGS); or the dsRBD is bound to the C terminus of the heavy chain of said Cetuximab antibody via said spacer peptide, and wherein said spacer peptide has an amino acid sequence of SEQ ID NO: 4 (GPGGGGSGGGGSGGGGS).

5. The recombinant protein of any one of the preceding claims, wherein said one or more dsRBm are selected from a group consisting of dsRBm of dsRNA dependent protein kinase (PKR), TRBP, PACT, Staufen, NFAR1, NFARZ, SPNR, RHA and NREBP.

6. The recombinant protein of any one of the preceding claims, wherein at least one of said one or more dsRBm is an amino acid sequence of a dsRNA dependent protein kinase (PKR), preferably a human PKR (hPKR).

7. The recombinant protein of any one of the preceding claims, wherein said dsRBD comprises amino acid residues 1-168 of human PKR or a homolog thereof, wherein in said homolog amino acid residues F10, F43, V45, I47, A71, V72, R39, F41, S59, K60, K61, K64, Y101, Y133, C135, M137, A161, F131, K150 and K154 are conserved, and wherein cysteine at position 121 and 135 of said dsRBD is exchanged by a non-cysteine amino acid, preferably said non-cysteine amino acid is selected from the group consisting of alanine, glycine, leucine, valine, 2-aminobutyric acid, norvaline, norleucine, isoleucine and allo-isoleucine; more preferably said non-cysteine amino acid is selected from the group consisting of alanine, glycine, leucine, valine, and isoleucine.

8. The recombinant protein of any one of the preceding claims, wherein said dsRBD comprises two double-stranded RNA-binding motifs (dsRBm), wherein one dsRBm (dsRBm1) consists of an amino acid sequence of residues 6-79 of hPKR or a homolog thereof; and the other dsRBm (dsRBm2) consists of an amino acid sequence of residues 96-169 of hPKR or a homolog thereof, wherein F10, F43, V45, I47, A71, V72, R39, F41, S59, K60, K61 and K64 are conserved in the homolog of dsRBm1, and Y101, Y133, C135, M137, A161, F131, K150 and K154 are conserved in the homolog of dsRBm2, wherein cysteine at position 121 and 135 of said dsRBD is exchanged by a non-cysteine amino acid, preferably said non-cysteine amino acid is selected from the group consisting of alanine, glycine, leucine, valine, 2-aminobutyric acid, norvaline, norleucine, isoleucine and allo-isoleucine; more preferably said non-cysteine amino acid is selected from the group consisting of alanine, glycine, leucine, valine, and isoleucine.

9. The recombinant protein of any one of the preceding claims, wherein said dsRBD has the sequence of SEQ ID NO: 8.

10. A complex comprising the recombinant protein of any one of claims 1 to 9 and a dsRNA.

11. The complex of claim 10, wherein said dsRNA is selected from the group consisting of polyinosinic-polycytidylic acid (polyIC), microRNA (miRNA), small interfering RNA (siRNA), small hairpin RNA (shRNA) and a combination thereof.

12. The complex of claim 10, wherein said dsRNA is polyinosinic-polycytidylic acid (polyIC).

13. A pharmaceutical composition comprising the recombinant protein or the complex of any one of the preceding claims and a pharmaceutically acceptable carrier.

14. The recombinant protein, complex or pharmaceutical composition of any one of the preceding claims for use in the treatment of cancer, wherein said cancer is characterized by EGFR-overexpressing cells.

15. A vector comprising a nucleic acid sequence encoding the recombinant protein of any one of claims 1 to 9.
